(19) United States
(12) Patent Application Publication
(10) Pub. No.: US 2017/0172979 A1
(43) Pub. Date: Jun. 22, 2017

(54) INHIBITORS OF LEUKOTRIENE-MEDIATED ACTIVITY FOR TREATING SIDE EFFECTS OF STATIN THERAPY

(71) Applicant: Yeda Research and Development Co. Ltd., Rehovot (IL)

(72) Inventors: Menachem RUBINSTEIN, Tel-Aviv (IL); Efrat DVASH-RIESENFELD, Rehovot (IL)

(21) Appl. No.: 15/118,196

(22) PCT Filed: Feb. 18, 2015
(86) PCT No.: PCT/IL2015/050184

§ 371 Date: Aug. 11, 2016

(30) Foreign Application Priority Data
Feb. 19, 2014 (IL) 231047
Feb. 19, 2014 (IL) 231048
May 28, 2014 (IL) 232851

(51) Int. Cl. A61K 31/366; A61K 31/381; A61K 31/41

(52) U.S. Cl. CPC A61K 31/366; A61K 31/41; A61K 31/381

(57) ABSTRACT

The present invention relates to the alleviation of adverse side effects resulting from statin therapy. The present invention further relates to the use of an inhibitor of leukotriene C₄ (LTC4) mediated activity for alleviating at least one side effects of statin therapy.

### FIELD OF THE INVENTION

The invention relates to methods for alleviation or reduction of side effects of statin therapy. Particularly, the present invention relates to compositions and kits comprising inhibitors of LTC4 mediated activity for attenuating at least one of the adverse side effects of statin therapy.

### BACKGROUND OF THE INVENTION

The endoplasmic reticulum (ER) is prone to stress by a broad range of physiological cues as well as toxic agents, typically leading to accumulation of misfolded ER client proteins. ER stress has been associated with many diseases, where it leads to cell death. Three ER stress sensor proteins. IRE1α. PERK and ATF6, located at the ER membrane, mediate an evolutionary conserved array of signaling pathways, termed the unfolded protein response (UPR). Initial UPR is aimed at coping with the stress by reducing the overload of misfolded proteins in the ER. Under excessive stress, the same UPR sensors trigger cell death. Several stress-triggered cell death mechanisms were identified, but the basis for toxicity of misfolded protein accumulation in the ER and the mechanisms involved are not completely understood.

A key player in stress-triggered cell death is the C/EBP-homologous protein CHOP (DDIT3, GADD153), which is induced by all three ER stress sensors. CHOP was shown to trigger apoptosis by down-regulating Bcl2 proteins and translocating Bax to the mitochondria. The stress-triggered TRAF2-ASK1-JNK pathway also triggers apoptosis by inhibiting Bcl2 proteins and activating Bim, BAX and BAC. However, in certain cell types and stress conditions, cell death occurs despite lack of Bcl-2 inhibition and ASK-1 or Bax/Bak activation, indicating the existence of additional death-triggering pathways. CHOP triggers cell death also through oxidative stress, thus, eliciting both apoptotic and non-apoptotic cell death mechanisms.

Studies with C. elegans demonstrated that ERO1, which generates H₂O₂ as a byproduct of protein disulfide bond formation in the ER, is the ROS producer under ER stress. However, in mouse cells this is not the case, as combined loss-of-function mutations in genes encoding all ER thiol oxidases ERO1α, ERO1β, and PRDX4 led to increased rather than reduced production of H₂O₂. Hence, the mechanism by which ER stress triggers oxidative stress has remained elusive. To identify an alternative mechanism, other ER oxidoreductases mechanisms were studied such as the structurally related enzymes Microsomal glutathione S-transferase 1 (MGST1) and MGST2. MGST1 was extensively studied as a pro-survival factor, conferring resistance to cytotoxic drugs both by direct detoxification and by downstream protection from oxidative stress. In contrast, the role of MGST2 in oxidative stress and ER stress has not been extensively studied

MGST2 is an isoenzyme of leukotriene C4 synthase (LTC4S). MGST2 is expressed mainly in mast cells and some other myeloid cells. Immunological cues such Fc receptor activation initiate LTC4 biosynthesis in mast cells by translocation to the nuclear membrane and co-localization of cytosolic phospholipase A2 (cPLA2), 5-lipoxygenase (5-LO), 5-lipoxygenase Activating Protein (FLAP) and LTC4S. cPLA2 releases arachidonic acid from phospholipids, 5-LO and FLAP oxidize it to LTA₄ and LTC4S conjugates LTA₄ with glutathione to form LTC4. LTC4 is then exported to the extracellular milieu by the transporter MRP1. Cell surface enzymes further metabolize LTC4 to the more stable forms LTD₄ (CAS number 73836-78-9) and LTE₄ (CAS number 75715-89-8). All three leukotrienes bind to two G-protein coupled receptors: CysLTR1 and CysLTR2. Secreted LTC4 and its metabolites trigger contraction of smooth muscle cells, thereby causing bronchoconstriction and vasoconstriction in the lungs, manifested as the classical symptoms of allergy and asthma. Therefore, several LTC4 receptor antagonists (montelukast (CAS number 158966-92-8; cyclopentyl 3-{2-methoxy-4-[(o-tolylsulfonyl)carbamoyl]benzyl}-1-methyl-1H-indol-5-ylcarbamate), pranlukast (CAS number 103177-37-3; N-[4-oxo-2-(1H-tetrazol-5-yl)-4H-chromen-7-yl]-4-(4-phenylbutoxy)benzamide), zafirlukast (CAS number 107753-78-6; cyclopentyl 3-{2-methoxy-4-[(o-tolylsulfonyl)carbamoyl]benzyl}-1-methyl-1H-indol-5-ylcarbamate), and cinalukast (CAS number 128312-51-6; 3-({3-[(E)-2-(4-cyclobutyl-1,3-thiazol-2-yl)ethenyl]phenyl}carbamoyl)-2,2-diethylpropanoic acid)) were developed and are approved drugs for the treatment of asthma symptoms.

Whereas LTC4S is expressed mainly in mast cells and has been extensively studied in the context of allergy and asthma, its isoenzyme MGST2 is ubiquitously expressed, but its physiological role has not been studied extensively.

Statins are competitive inhibitors of 3-hydroxy-3-methyl glutaryl coenzyme A reductase (HMG-CoA reductase, HMGCR, UniProt P04035), the rate-limiting enzyme of cholesterol biosynthesis, which serve as the primary therapy for hypercholesterolemia and for preventing cardiovascular diseases. The therapeutic effects of statins for managing cholesterol, including the primary outcome of reduced atherosclerosis, are well established. To lower high cholesterol levels, or to prevent increased cholesterol levels in patients of risk, patients are treated with a range of statins, which include atorvastatin (CAS number 134523-00-5), simvastatin (CAS number 79902-63-9), pravastatin (CAS number 81093-37-0), lovastatin (CAS number 75330-75-5) and fluvastatin (CAS number 93957-54-1). Cerivastatin (CAS number 145599-86-6) was marketed in the late 1990s, but was voluntarily withdrawn from the market worldwide in 2001, due to reports of fatal rhabdomyolysis (a condition in which damaged skeletal muscle tissue breaks down).

Additionally, statins have a number of pleiotropic, cholesterol-independent effects related to endothelial function, insulin sensitivity, and inflammation/immunomodulation. The statins have also been reported to have potential utility in the treatment of dementia and various cancers, e.g. prostate, skin, lung, colon, bladder, uterus and kidney.

However, there are a number of potentially serious side effects associated with statin therapy, including myopathies, that may range in severity from myositis to muscle wastage (rhabdomyolysis). Other less serious adverse effects have been reported, including headache, joint pain, fever, back pain, abdominal cramping, sleep disorder, rhinitis, sinusitis, stimulation of coughing reflex, dizziness and fatigue. Of the contraindications reported for this group of drugs, two of the most common are fatigue and/or muscle pain (often referred to as “myalgia”). The risk of adverse side effects during treatment with the statins is increased with concurrent administration of certain other drugs, such as cyclosporin, fabric acid derivatives (e.g. gemfibrozil), niacin or antifungal drugs.

In fact, an estimated 5-10% of patients discontinue statin use due to myopathic symptoms ranging from mild to moderate myalgia characterized by muscle weakness, fatigue, and pain, to life-threatening rhabdomyolysis, which is defined as a massive and acute destruction of muscle fibers resulting in the release of muscle fiber contents. Reports of myositis (muscle inflammation) and myopathic symptoms increase with increased statin dose, with different classes of statins, when statins are coupled with other drugs or with exercise.

Various hypotheses have been proposed to explain statin-induced muscle injury. Statin effect can be indirect through the reduction of cholesterol synthesis or a direct effect on different muscle cell targets. The mechanistic underpinnings of statin myopathy is likely multifactorial and partially attributed to the regulatory effects of statins on apoptosis. However, how statins promote apoptosis is fairly obscure. In intact cells, statins were found to trigger elevation of calcium levels, translocation of Bax, a pro-apoptotic protein to the mitochondria, the mitochondrial permeability transition pore (PTP) to open, and cytochrome C to be released, resulting in apoptosis.

In addition to apoptosis, several studies in vitro and in vivo demonstrated that statins trigger both oxidative stress and necrosis. Most importantly, several statins induced endoplasmic reticulum (ER) stress, characterized by the induction of the protein CHOP (CCAAT/-enhancer-binding protein homologous protein), which triggers cell death also through oxidative stress, eliciting both apoptotic and non-apoptotic cell death mechanisms. Acute application of statins has also been shown to trigger a massive calcium release from the endoplasmic reticulum (ER) via ryanodine receptors. Calcium release from the ER is a hallmark of ER stress and ER stress triggers oxidative stress. Altogether, statin-triggered myopathy involves at least in part ER stress-triggered oxidative stress, leading to cell death.

Leukotrienes are a family of eicosanoid inflammatory mediators produced in leukocytes by the oxidation of arachidonic acid by the enzyme arachidonate 5-lipoxygenase (5-lipoxygenase, 5-LOX, 5-LO, UniProtP 09917). As their name implies, leukotrienes were first discovered in leukocytes, but have since been found in other immune cells. Leukotrienes use lipid signaling to convey information to either the cell producing them (autocrine signaling) or neighboring cells (paracrine signaling) in order to regulate immune responses. Leukotrienes production is usually accompanied by the production of histamine and prostaglandins, which also act as inflammatory mediators. One of their roles (specifically, leukotriene D4) is to trigger contractions in the smooth muscles lining the bronchioles; their overproduction is a major cause of inflammation in asthma and allergic rhinitis. Leukotriene antagonists are used to treat these disorders by inhibiting the production or activity of leukotrienes.

Leukotrienes are roughly divided into three types. LTC4 (CAS number 72025-60-6), LTD4, LTE4 (CAS number 75715-89-8) and LTF4 (CAS Number 83851-42-7) are often called “cysteinyl leukotrienes” due to the presence of the amino acid cysteine in their structure. The cysteinyl leukotrienes make up the slow-reacting substance of anaphylaxis (SRS-A). LTF4, like LTD4, is a metabolite of LTC4, but, unlike LTD4, which lacks the glutamic residue of glutathione, LTF4 lacks the glycine residue of glutathione. LTB₄ is synthesized in vivo from LTA₄ by the enzyme LTA₄ hydrolase. Its primary function is to recruit neutrophils to areas of tissue damage, though it also helps promote the production of inflammatory cytokines by various immune cells. Drugs that block the actions of LTB₄ have shown some efficacy in slowing the progression of neutrophil-mediated diseases. There has also been postulated the existence of LTG₄, a metabolite of LTE₄ in which the cysteinyl moiety has been oxidized to an alpha-keto-acid (i.e.—the cysteine has been replaced by a pyruvate).

Receptors of leukotrienes are classified pharmacologically into three types, namely the BLT receptor, CysLT1 receptor (UniProt Q9Y271), and CysLT2 receptor (UniProt Q9NS75). The BLT receptor specifically recognizes the LTB4. The CysLT1 receptor and CysLT2 receptor both recognize peptide leukotrienes, namely, leukotriene C4 (LTC4), leukotriene D4 (LTD4), and leukotriene E4 (LTE4).

Thompson et al., (“Statin-associated myopathy”, JAMA, 2003, Vol. 289(13):1681-90) performed a literature review to provide a clinical summary of statin-associated myopathy and discuss possible mediating mechanisms. The literature review found that reports of muscle problems during statin clinical trials are rare, cerivastatin being the most commonly implicated statin. The review further notes that it is not clear how statins injure skeletal muscle.

Thus, there is an unmet need for compositions and methods for preventing or ameliorating major side effects associated with statin therapy.

### SUMMARY OF THE INVENTION

The present invention provides pharmaceutical compositions, methods and kits for treating one or more serious adverse side effects of statin therapy. Particularly, the present invention provides a pharmaceutical composition comprising an effective amount of at least one inhibitor of leukotriene C4 (LTC4)-mediated activity for attenuating one or more adverse side effects of statin therapy.

The present invention is based, in part, on the unexpected and surprising finding that LTC4 receptor antagonists, in particular antagonists of cysteinyl leukotriene receptor 2, are capable of attenuating the adverse side effects of HMGCoA (hydroxy-3-methyl glutaryl coenzyme A) reductase inhibitors, known as statins, while maintaining the desired effects thereof. The present invention is further based, in part, on the unexpected and surprising finding that antagonists of cysteinyl leukotriene receptor 2 (CysLTR2) are capable of attenuating the adverse side effects of statins in cultures of myocytes. In certain non-myocyte cell lines antagonists of cysteinyl leukotriene receptor 1 (CysLTR1) also diminished the toxicity of statins. However, without wishing to be bound to any theory or mechanism, antagonists of CysLTR1 may still be utilized to protect a variety of cell types, including myocytes, from adverse effects of statins, in view of the efficacy of BAY-u9773, a dual-specific antagonist of CysLTR1 and CysLTR2, demonstrated in the Examples section below.

The present invention discloses a previously unrecognized general signaling cascade, activated by ER stress-triggering agents, which plays a significant role in initiating cell death. Based on the newly discovered signaling cascade, the present invention provides means for attenuating statin-triggered ER stress, thus treating one or more adverse side effects of statin therapy.

Statins trigger necrosis, which leads to release of the nuclear protein HMGB1. HMGB1 release recruits immune cells such as macrophages and granulocytes, which trigger local inflammation and further tissue damage. Without being linked to any theory or mechanism, LTC4 inhibitors will attenuate the initial necrosis induced by statins. Hence. LTC4 inhibitors do not act as inhibitors of inflammation, in particular muscle inflammation, but rather as inhibitors of cell death e.g. by necrosis. It is therefore understood that LTC4 inhibitors prevent the formation of inflammation rather than treating the inflammation. The present invention suggests, for the first time, to use LTC4 inhibitors as prophylactic or treatment for muscle damage or muscle inflammation.

According to one aspect, the present invention provides a pharmaceutical composition comprising at least one statin, and at least one agent selected from the group consisting of an antagonist of CysLTR2, and an inhibitor of LTC4 biosynthesis.

In certain embodiments, the statin is selected from the group consisting of atorvastatin, fluvastatin, lovastatin, pitavastatin, pravastatin, rosuvastatin, and simvastatin, and any combination thereof. Each possibility represents a separate embodiment of the present invention.

In certain embodiments, the agent is an antagonist of CysLTR2. According to some embodiments, the CysLTR2 antagonist is selected from the group consisting of: BAY-cysLT2 (CAS Number 712313-33-2), BAY-u9773 (CAS Number 154978-38-8). HAMI3379 (CAS Number 712313-35-4), and any combination thereof. Each possibility represents a separate embodiment of the present invention. In certain embodiments, the antagonist of CysLTR2 inhibits the activity of CysLTR2 and the activity of cysteinyl leukotriene receptor 1 (CysLTR1). In certain embodiments, the agent is an inhibitor of LTC4 biosynthesis. In certain embodiments, the inhibitor of LTC4 biosynthesis inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP). In certain embodiments, the inhibitor of LTC4 biosynthesis is zileuton. In certain embodiments, the inhibitor of 5-LO is atreleuton (CAS number 154355-76-7). In certain embodiments, the inhibitor of FLAP is MK-886 (CAS number 118414-82-7). In certain embodiments, the pharmaceutical composition described above comprises a statin, an antagonist of CysLTR2, and an inhibitor of LTC4 biosynthesis.

In certain embodiments, the pharmaceutical compositions described above are for use in preventing or reducing an adverse side effect induced by a statin in muscle tissue of a subject, wherein the subject is receiving statin therapy and experiencing or at risk of experiencing an adverse side effect induced by the statin.

According to certain embodiments, the adverse side effect is a statin-induced myopathy. In certain embodiments, the myopathy is selected from the group consisting of myositis and rhabdomyolysis. Each possibility represents a separate embodiment of the invention.

The present invention provides, in another aspect, a method for preventing or reducing an adverse side effect induced by a statin in muscle tissue of a subject, comprising the steps of administering to the subject at least one statin and at least one agent selected from the group consisting of an antagonist of CysLTR2 and an inhibitor of LTC4 biosynthesis, wherein the subject is receiving statin therapy and experiencing or at risk of experiencing an adverse side effect induced by the statin According to certain embodiments, the adverse side effect is a statin-induced myopathy.

In certain embodiments, the myopathy is selected from the group consisting of myositis and rhabdomyolysis. Each possibility represents a separate embodiment of the invention.

The present invention further provides, in another aspect, a kit comprising a pharmaceutical composition comprising at least one statin, and a pharmaceutical composition comprising at least one agent selected from the group consisting of an antagonist of CysLTR2 and an inhibitor of LTC4 biosynthesis.

In certain embodiments, the statin is selected from the group consisting of atorvastatin, fluvastatin, lovastatin, pitavastatin, pravastatin, rosuvastatin, and simvastatin, and any combination thereof. Each possibility represents a separate embodiment of the present invention.

In certain embodiments, the agent is an antagonist of CysLTR2. According to some embodiments, the CysLTR2 antagonist is selected from the group consisting of: BAY-cysLT2 (CAS Number 712313-33-2), BAY-u9773 (CAS Number 154978-38-8). HAMI3379 (CAS Number 712313-35-4), and any combination thereof. Each possibility represents a separate embodiment of the present invention. In certain embodiments, the antagonist of CysLTR2 inhibits the activity of CysLTR2 and the activity of cysteinyl leukotriene receptor 1 (CysLTR1). In certain embodiments, the agent is an inhibitor of LTC4 biosynthesis. In certain embodiments, the inhibitor of LTC4 biosynthesis inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP). In certain embodiments, the inhibitor of LTC4 biosynthesis is zileuton. In certain embodiments, the inhibitor of 5-LO is atreleuton (CAS number 154355-76-7). In certain embodiments, the inhibitor of FLAP is MK-886 (CAS number 118414-82-7). In certain embodiments, the kit described above comprises a pharmaceutical composition comprising a statin, a pharmaceutical composition comprising an antagonist of CysLTR2, and a pharmaceutical composition comprising an inhibitor of LTC4 biosynthesis.

In certain embodiments, the kits described above further comprise instructions for administering the statin and the agent to a subject receiving statin therapy and experiencing or at risk of experiencing an adverse side effect induced by the statin. In certain embodiments, the kits described above are for use in preventing or reducing an adverse side effect induced by a statin in muscle tissue of a subject, wherein the subject is receiving statin therapy and experiencing or at risk of experiencing an adverse side effect induced by the statin. In certain embodiments, the adverse side effect is a statin-induced myopathy. In certain embodiments, the myopathy is selected from the group consisting of myositis and rhabdomyolysis. In certain embodiments, the use comprises administering the agent prior to, during, and/or after administering the statin to the subject. Each possibility represents a separate embodiment of the invention.

The present invention further provides, in an aspect, a pharmaceutical composition comprising at least one antagonist of cysteinyl leukotriene receptor 2 (CysLTR2) for use in preventing or reducing an adverse side effect induced by a statin in muscle tissue of a subject, wherein the subject is receiving statin therapy and experiencing or at risk of experiencing an adverse side effect induced by the statin.

According to another aspect, the present invention provides a pharmaceutical composition comprising at least one inhibitor of LTC4 biosynthesis for use in preventing or reducing an adverse side effect induced by a statin in muscle tissue of a subject, wherein the subject is receiving statin therapy and experiencing or at risk of experiencing an adverse side effect induced by the statin, and wherein the at least one inhibitor of LTC4 biosynthesis inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP).

According to certain embodiments, the adverse side effect is a statin-induced myopathy. In certain embodiments, the myopathy is selected from the group consisting of myositis and rhabdomyolysis. Each possibility represents a separate embodiment of the invention.

In certain embodiments, the statin is selected from the group consisting of atorvastatin, fluvastatin, lovastatin, pitavastatin (CAS number 147511-69-1), pravastatin, rosuvastatin (CAS number 287714-41-4), and simvastatin, and any combination thereof. Each possibility represents a separate embodiment of the present invention.

In certain embodiments, the antagonist of CysLTR2 inhibits the activity of CysLTR2 and the activity of cysteinyl leukotriene receptor 1 (CysLTR1). According to some embodiments, the CysLTR2 antagonist is selected from the group consisting of: BAY-cysLT2 (CAS Number 712313-33-2), BAY-u9773 (CAS Number 154978-38-8), HAMI3379 (CAS Number 712313-35-4), and any combination thereof. Each possibility represents a separate embodiment of the present invention.

In certain embodiments, the pharmaceutical compositions described comprises a statin, an antagonist of CysLTR2 and an inhibitor of LTC4 biosynthesis which inhibits the activity of an enzyme selected from the group consisting of MGST2, cPLA2, 5-LO, and FLAP. Each possibility represents a separate embodiment of the invention. In certain embodiments, the inhibitor of 5-LO is zileuton. In certain embodiments, the inhibitor of 5-LO is atreleuton. In certain embodiments, the inhibitor of FLAP is MK-886.

In certain embodiments, the pharmaceutical composition described above comprise an antagonist of CysLTR2 and an inhibitor of LTC4 biosynthesis which inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP).

According to some embodiments, the pharmaceutical compositions described above further comprises an effective amount of statin selected from the group consisting of atorvastatin, cerivastatin, fluvastatin, lovastatin, mevastatin, pitavastatin, pravastatin, rosuvastatin, simvastatin, and any combination thereof. Each possibility represents a separate embodiment of the present invention.

According to additional embodiments the dosages used for statin is in a range similar to that of the approved drugs when given separately for their approved indications.

The present invention provides, in another aspect, a method for preventing or reducing an adverse side effect induced by a statin in muscle tissue of a subject, comprising the step of administering at least one antagonist of CysLTR2 to the subject, wherein the subject is receiving statin therapy and experiencing or at risk of experiencing an adverse side effect induced by the statin.

The present invention provides, in yet another aspect, a method for preventing or reducing an adverse side effect induced by a statin in muscle tissue of a subject, comprising the step of administering to the subject at least one inhibitor of LTC4 biosynthesis, wherein the subject is receiving statin therapy and experiencing or at risk of experiencing an adverse side effect induced by the statin, and wherein the inhibitor of LTC4 biosynthesis inhibits the activity of an enzyme selected from the group consisting of MGST2, cPLA2, 5-LO, and FLAP.

In certain embodiments, the adverse side effect is a statin-induced myopathy. In certain embodiments, the adverse side effect is a statin-induced myopathy.

In certain embodiments of the methods described above, the inhibitor of leukotriene mediated activity is administered to the subject being treated by the statin prior to the treatment by the statin, at the same time with the treatment by the statin, or together with the treatment by the statin. In certain embodiments of the methods described above, the inhibitor of leukotriene mediated activity is administered to the subject being treated by the statin after the treatment by the statin. Each possibility represents a separate embodiment of the invention.

In some embodiments, the present invention provides a method for treating one or more side effects of statin therapy, comprising administering to a subject undergoing statin therapy, an effective amount of an inhibitor of leukotriene mediated activity. According to some embodiments, the inhibitor of leukotriene mediated activity can be administered alone or in conjunction with an effective amount of a statin.

Any suitable route of administration to a subject may be used for the composition of the present invention. The preferred mode of administration will depend upon the particular indication being treated and will be apparent to one of skill in the art.

According to some embodiments, the pharmaceutical compositions of the present invention may be provided as a pill for oral administration.

The combination of components constituting the treatment may be administered either simultaneously (as discrete dosage forms or as a single composition), sequentially, or separated by a suitable time interval. Each possibility represents a separate embodiment of the present invention. Where the components are administered as discrete dosage forms, i.e., not as combined compositions, each component may be administered in the same form or a different form, e.g., oral, nasal, parenteral, or dermal. When the compounds are administered simultaneously, sequentially or separately, the components may be provided as discrete dosage forms.

Where the components are administered as discrete dosage forms, i.e., not as intimate compositions, each component may be administered in the same form or a different form, e.g., oral, nasal, parenteral, rectal, vaginal or dermal. When the compounds are administered simultaneously, sequentially or separately, the components may be provided as discrete to dosage forms. Optionally the components of the combination may be provided in a kit form wherein the kit is preferably in compartmentalized form adapted for the discrete administration of the components.

Alternatively, when the components of the combination are administered simultaneously, they may be provided as a single composition containing the two or more components or may be provided in a kit form, wherein the kit is compartmentalized for the simultaneous administration of the components.

Where the pharmaceutical composition of inhibitor of leukotriene mediated activity and the therapeutic statin drug are administered as discrete dosage forms, each may be formulated together with one or more pharmaceutically acceptable carrier to form compositions. Where the components of the therapy are administered as a single composition, the composition may also optionally comprise one or more pharmaceutically acceptable carrier.

The formulation of pharmaceutical compositions is well known to those skilled in the art. Such compositions may contain any suitable carriers such as, diluents or excipients, which are pharmaceutically acceptable in the sense of being compatible with the other ingredients of the composition and not injurious to the subject.

Suitable carriers include all conventional solvents, oils, dispersion media, fillers, solid carriers, coatings, antifungal and antibacterial agents, dermal penetration agents (where appropriate), surfactants, isotonic and absorption agents and the like. It will be understood that the compositions of the invention may also include other supplementary physiologically active agents.

The compounds and/or the pharmaceutical compositions of the current invention may be administered by any suitable method known in the art. For example, in a non-limiting manner, it can be administered locally, parenterally, orally, intra-nasally, intravenously, intramuscularly, subcutaneously, or by other suitable means. Each possibility represents a separate embodiment of the present invention. In a preferred embodiment the compounds and/or compositions of the current invention may be suitably formulated for oral administration (although other forms may, under appropriate circumstances, also be contemplated) and may be formulated in a discrete units form selected from the group consisting of: discrete units such as capsules, sachets of powders, granules and tablets. Each possibility represents a separate embodiment of the present invention. According to another embodiment the compound and/or compositions of the invention may be formulated in a form selected from the group consisting of powder, granules, solution, suspension in an aqueous or non-aqueous liquid, oils, paste, an oil-in-water liquid emulsion and water-in-oil liquid emulsion. Each possibility represents a separate embodiment of the present invention. In a preferred embodiment the pharmaceutical composition is formulated as single pills containing both statin and an inhibitor of leukotriene mediated activity for oral administration.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant (e.g. sodium starch glycolate, cross-linked polyvinyl pyrrolidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach. The compounds may also be presented in the form of hard or soft gelatin capsules. It should be understood that in addition to the active ingredients particularly mentioned above, the compositions of this invention may include other agents or additives conventional in the art having regard to the type of composition in question, for example, those suitable for oral administration may include such further agents as binders, sweeteners, thickeners, flavoring agents disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Each possibility represents a separate embodiment of the present invention.

According to some embodiments, use of the combination therapy with the pharmaceutical composition of the present invention will provide similar cholesterol lowering effect as that of the said statin when administered as a single therapy, while reducing its side effects.

According to some embodiments, the pharmaceutical compositions described above further comprise a pharmaceutically acceptable carrier.

Other objects, features and advantages of the present invention will become clear from the following description and drawings.

### BRIEF DESCRIPTION OF THE FIGURES

FIG. 1. Schematic description of the mechanism of ER stress triggered cell death.

FIGS. 2A-2B. ER stress regulates expression of MGST2 and 5-LO. (2A) Kinetics of microsomal glutathione S transferase (MGST2) and cleaved caspase 3 (Casp. 3) expression following treatment of human WISH epithelial cells and human HaCaT pre-keratinocytes with tunicamycin (1 μg/ml, 24 hours), as determined by immunoblotting. (2B) Kinetics of 5-lipoxygenase (5-LO) expression following treatment of human WISH epithelial cells with Brefeldin A (0.66 μg/ml, 24 hours) and mouse B16 cells with tunicamycin (1 μg/ml, 24 hours), as determined by immunoblotting.

FIG. 3. Kinetics of CysLTR1 and CysLTR2 expression following treatment of human WISH cells with Brefeldin A (0.66 μg/ml, 24 hours), and mouse B16 cells with tunicamycin (1 μg/ml, 24 hours), as determined by immunoblotting.

FIGS. 4A-4E. ER stress-triggered ROS accumulation is mediated by MGST2-derived LTC4. (4A) Mouse B16 cells were transfected with control siRNA or MGST2 siRNA, then treated with tunicamycin and stained with the superoxide anion indicator dihydroethidium (DHE). Quantitation of relative DHE fluorescence intensity is presented as determined by the ImageJ program. N=3. ***p<0.001. (4B) Mouse B16 cells were transfected with control siRNA or MGST2 siRNA, then treated with tunicamycin and stained with the superoxide anion indicator dihydroethidium (DHE). Silencing efficiency of mouse MGST2 mRNA as determined by qRT-PCR on replicate samples is shown. N=3, ***p<0.001. (4C) Human HaCaT pre-keratinocytes were treated with Brefeldin A (0.33 μg/ml, 24 hours) with or without the MRP1 inhibitor reversan (20 μM) or the LTC4 antagonists pranlukast, BAY-cysLT2 or BAY-u9773. The cells were then stained with the ROS indicator DCFH-DA. Quantitative analysis of relative DCF fluorescence intensity is presented. All inhibitors except reversan significantly inhibited ROS accumulation. N=3, * p<0.05. (4D) Immunoblotting with anti FLAG tag of HEK 293T cell extract following transfection with either control pcDNA4 vector or pcMGST2-FLAG. (4E) HEK 293T cells were either mock-transfected (Control), transfected with pcDNA4 or with pcMGST2-FLAG and then stained with DHE. Quantitative analysis of relative DHE fluorescence intensity is presented N=4, **p<0.01.

FIG. 5. ER stress-triggered ROS accumulation is mediated by LTC4. Mouse B16 cells were treated with tunicamycin (1 μg/ml, 24 hours) with or without BAY-u9773 (1 μM) or montelukast (5 μM). After 24 hours the cells were stained with DCF-DA (10 μM, 40 min). Quantitative analysis of relative DCF fluorescence intensity is presented. The average intensity of DCF was normalized to that of the nuclei staining using the Photoshop histogram function. The data are average±SD of measuring three fields. (**p<0.01).

FIGS. 6A-6C. ER stress-triggered NADPH oxidase 4 (NOX4) expression, translocation towards the nuclei and DNA oxidation are mediated by the MGST2-LTC4 pathway. (6A) Human WISH cells were treated with vehicle (Control) or Brefeldin A (0.66 μg/ml, 24 hours) in the absence or presence of BAY-u9773, MK571 or Montelukast. NOX4 levels were evaluated by immunoblotting. (6B) Mouse B16 cells were treated with vehicle (Control) or tunicamycin (0.5 μg/ml, 24 hours) in the absence or presence of Montelukast, BAY-u9773 or BAY-cysLT2. NOX4 levels were evaluated by immunoblotting. (6C) Human HaCaT pre-keratinocytes were treated with vehicle (Control) or Brefeldin A (48 hours) in the absence or presence of pranlukast or BAY-u9773, then immunostained with anti 8-OHdG. Quantitative analysis of 8-OHdG fluorescence intensity is shown. N=3 *p<0.05, ****p<0.001.

FIGS. 7A-7G. The MGST2-LTC4 pathway mediates ER stress-triggered cell death. (7A) Mouse B16 cells were transfected with control siRNA (siControl) or MGST2-specific siRNA (siMGST2), treated with vehicle or tunicamycin (1 μg/ml, 24 hours) (Tm) and then stained with Crystal violet. Relative viability of the cells is shown. N=3 ***p<0.0001. (7B) Mouse B16 cells were transfected with control siRNA (siControl) or MGST2-specific siRNA (siMGST2), treated with vehicle or tunicamycin (Tm) and then stained with Crystal violet. Silencing efficiency of mouse MGST2 mRNA as determined by qRT-PCR on replicate samples is shown. N=3, ***p<0.001. (7C) HEK 293T cells were mock-transfected, transfected with pcDNA4 or with pcMGST2-FLAG, treated with vehicle (Control) or Brefeldin A (0.66 μg/ml, 24 hours) and then stained with Crystal violet. Relative viability of the cells is shown. N=3 ****p<0.0001. (7D) Human HaCaT pre-keratinocytes were treated with vehicle (Control) or Brefeldin A (BfA, 1.33 μg/ml, 48 hours) in the absence or presence of BAY-u9773 (80 nM). The plates were then stained with Crystal violet. Relative viability of the cells is shown. N=4 ***p<0.0001. (7E) Human WISH cells were treated either with vehicle (Control) or tunicamycin (1 μg/ml, 48 hours) in the absence or presence of BAY-u9773 (80 nM) and then stained with Neutral red. Relative viability of the cells is shown. N=4 ***p<0.0001. (7F) Human HaCaT pre-keratinocytes were treated with vehicle (Control) or MG262 (0.05 μM) in the presence or absence of zileuton (10 μM, 24 hours). The plates were then stained with Crystal violet. Relative viability of the cells is shown. N=4, ***p<0.001. (7G) Human WISH cells were treated with vehicle (Control) or Brefeldin A (0.66 μg/ml, 24 hours) in the absence or presence of pranlukast (10 μM). The plates were then stained with Crystal violet. Relative viability of the cells is shown. N=3, ***p<0.001.

FIGS. 8A-8C. Inhibitors of the MGST2-LTC4 Pathway Attenuate ER Stress-triggered Cell Death. (8A) Human WISH cells were treated with vehicle (Control) or Brefeldin A (0.66 μg/ml, 48 hours) in the absence or presence of BAY-cysLT2. The plates were then stained with Crystal violet. Relative viability of the cells is shown. N=4, ***p<0.001. (8B) Mouse B16 cells were treated with vehicle (Control) or tunicamycin (1 μg/ml, 24 hours) (Tm), thapsigargin (50 nM, 24 hours) (Tg) or Brefeldin A (BfA 1.3 μM, 24 hours) in the presence or absence of MK571 for 24 h. The plates were stained with Crystal violet. Relative viability of the cells is shown. N=4. ***p<0.001. (8C) Immunoblot of the necrosis marker HMGB1 released to the culture media of B16 cells treated with vehicle (Control) or Brefeldin A (1.3 μg/ml, 24 hours) in the presence or absence of MK571. Ponceau staining serves as loading control.

FIGS. 9A-9G. MGST2 Deficiency attenuates ER Stress-Triggered Death in Vivo. (9A) Agarose Gel electrophoresis of PCR products obtained by PCR of DNA from tail-ends of WT and homozygous MGST2-deficient (KO) 129svEvBrd mice. DNA of heterozygous ES cells (ES) and negative PCR control are shown as well. (9B) Fibroblasts of WT and MGST2-deficient mouse embryos at passage 2 were treated with vehicle (Control) or tunicamycin (1 μg/ml, 24 hours). The plates were then stained with Crystal violet. Relative viability as determined by neutral red staining of WT and MGST2-deficient mouse embryos at passage 2, treated with vehicle (Control) or tunicamycin is shown. (9C) Brefeldin A induction of apoptosis as determined by immunoblotting of cleaved caspase 3 (Casp. 3) in WT and in MGST2 deficient MEFs. (9D) Fibroblasts of WT and MGST2-deficient mouse embryos at passage 2 were treated with vehicle (Control) or tunicamycin (2 μg/ml, 24 hours) or Brefeldin A (0.25 μg/ml, 24 hours) and then stained with DCFH-DA. Quantitative analysis of DCF fluorescence intensity is shown. N=3 *** p<0.001. (9E) Tunicamycin (1 mg/kg) was administered ip once at time=0 to WT and MGST2-deficient mice (5/group). Kidneys were excised and slices were stained with hematoxylin-eosin. Quantitative analysis of damage to kidney proximal tubules is shown. Images of entire kidney's areas containing the proximal tubules were selected using the lasso tool of Photoshop and vacuoles were counted using the ImageJ program. N=5, ***p<0.03. (9F) Survival of WT and MGST2-deficient mice (20/group) to which tunicamycin (2.5 mg/kg) was administered ip once at time=0. Mice showing severe morbidity were euthanized to reduce suffering. (9G) Survival of WT mice (4/group) to which tunicamycin (2.5 mg/kg) was administered ip once at time-0 and either vehicle (Control) or pranlukast (1 mg/kg/day, 3 days). Mice showing severe morbidity were euthanized to reduce suffering.

FIG. 10. Pranlukast attenuates simvastatin triggered cell death. Survival of human WISH cells treated with the indicated concentrations of simvastatin in the presence (dashed line) or absence (continuous line) of pranlukast (10 μM, 48 hours). The plates were then stained with Crystal violet and the relative cell viability was determined.

FIGS. 11A-11B. BAY-cysLT2 and BAY-u9773 but not pranlukast attenuate simvastatin-triggered cell death of differentiated C2C12 mouse myocytes. (11A) Survival of mouse C2C12 cells following differentiation into myocytes, treated with 10 uM simvastatin for 5 days in the presence of vehicle, pranlukast (10 μM), BAY-cysLT2 (10 μM), BAY-u9773 (1 μM) or Mevalonate (71.4 μM). The plates were then stained with Crystal violet and the relative cell viability was determined. (11B) Quantification of the staining intensity is shown.

FIGS. 12A-12B. Zileuton but not montelukast attenuates simvastatin-triggered death of differentiated C2C12 mouse myocytes. (12A) C2C12 immortalized mouse myoblasts (15,000 cells/100 ul DMEM) were seeded for 24 and then differentiated for 3 days (initiated by serum free medium supplemented with 1×ITS medium) and then treated with 20 μg/ml Simvastatin with or without montelukast (2 μM), zileuton (10 μM) or mevalonate (71.4 μM). After 4 days the cells were stained with crystal violet and photographed under light microscope. (12B) Quantification of the staining intensity is shown.

### DETAILED DESCRIPTION OF THE INVENTION

The present invention provides methods, pharmaceutical compositions and kits for treating one or more adverse side effects of statin therapy. Particularly, the present invention provides a pharmaceutical composition comprising an effective amount of at least one inhibitor of leukotriene mediated activity for attenuating one or more adverse side effects of statin therapy.

The inventors of the present invention have revealed a previously unrecognized general signaling cascade, activated by ER stress-triggering agents, which plays a significant role in initiating cell death. Based on the newly discovered signaling cascade, the present invention provides means for attenuating statin-triggered ER stress, thus treating one or more adverse side effects of statin therapy.

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The present invention reveals for the first time a previously unrecognized general signaling cascade, activated by ER stress-triggering statins, which plays a significant role in initiating cell death in statin-receiving patients. The present invention provides means to attenuate statin-triggered ER stress by inhibiting said signaling cascade. In addition, the present invention discloses MGST2-LTC4 as a previously unrecognized signaling cascade, activated by ER stress-triggering agents, as well as by statins.

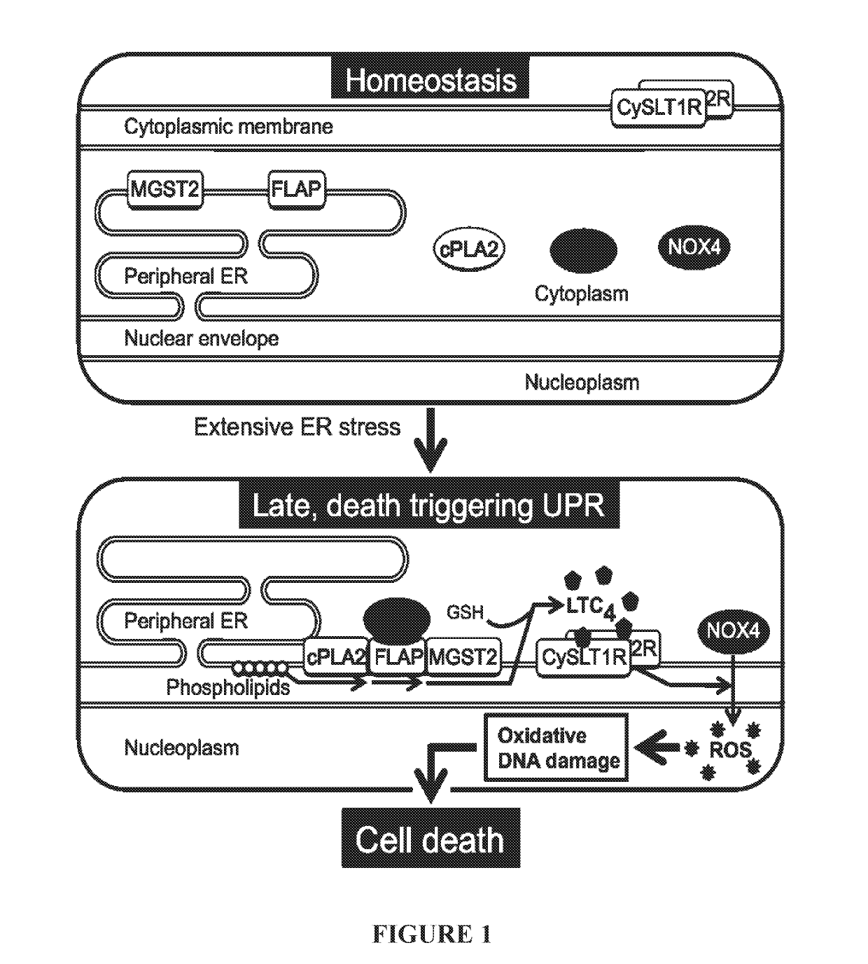

The present invention is based, in part, on the following unexpected discoveries: (a) ER stress, elicited by specific reagents such as tunicamycin, thapsigargin and Brefeldin A, triggers cell death at least in part through generation of leukotriene C (LTC4); (b) This LTC4 is generated by the enzyme MGST2, and ER stress activates MGST2 by its co-translocation to the nuclear envelope and its co-localization together with cPLA2, 5LO and FLAP; (c) ER stress also triggers the translocation of the two LTC4 receptors, CysLT1 and cysLT2, to the nuclear envelope and their co-localization with the synthetic machinery of LTC4, thereby enabling localized intracrine action of LTC4; (d) Binding of the LTC4 to its internalized receptors activates NADPH oxidase 4, resulting in ROS accumulation, and LTC4 is the major trigger of oxidative stress due to ER stress; and (e) ROS accumulation mediated by the ER stress-activated MGST2-LTC4 pathway leads to DNA damage and subsequent cell death (FIG. 1). The LTC4 biosynthetic machinery and its receptors translocate and co-localize at the nuclear envelope in response to ER stress, generally termed “unfolded protein response” (UPR). As a result NOX4 is activated, generating ROS, which inflict oxidative DNA damage and subsequent cell death. Thus, the present invention discloses a major death-triggering pathway, activated by ER stress.

The present invention further discloses that the LTC4 receptor antagonists such as montelukast and pranlukast attenuated the toxicity of statins such as simvastatin as well as atorvastatin, cerivastatin, fluvastatin, lovastatin, mevastatin (CAS number 73573-88-3), pitavastatin, pravastatin and rosuvastatin. The present invention further discloses that LTC4 receptor antagonists protect wild type mice from ER stress-triggered morbidity. Several LTC4 receptor antagonists (montelukast, pranlukast, etc.) were developed and are approved drugs for the treatment of asthma symptoms. All LTC4 receptor antagonists approved as drugs are selective inhibitors of the CysLT1 receptor. Selective inhibitors were also developed for CysLT2, the second LTC4 receptor, but so far they were not subjected to clinical development. Earlier studies demonstrated that CysLTR1 and CysLTR2, the two LTC4 receptors, are not equally expressed in different tissue types. Hence, the present invention also provides Cys LT2 receptor antagonists as well as a combination of cysLT1 and CysLT2 receptor antagonists for alleviating some of the major adverse side effects of statins. The present invention also discloses use of non-selective LTC4 receptor antagonists for alleviating some of the adverse side effects of statins. The present invention further provides inhibitors of leukotriene biosynthesis for alleviating some of the adverse side effects of statins.

According to one aspect, the present invention provides a pharmaceutical composition comprising at least one statin, and at least one agent selected from the group consisting of an antagonist of CysLTR2, and an inhibitor of LTC4 biosynthesis.

The present invention provides, in another aspect, a method for preventing or reducing an adverse side effect induced by a statin in muscle tissue of a subject, comprising the steps of administering to the subject at least one statin and at least one agent selected from the group consisting of an antagonist of CysLTR2 and an inhibitor of LTC4 biosynthesis, wherein the subject is receiving statin therapy and experiencing or at risk of experiencing an adverse side effect induced by the statin.

The present invention further provides, in another aspect, a kit comprising a pharmaceutical composition comprising at least one statin, and a pharmaceutical composition comprising at least one agent selected from the group consisting of an antagonist of CysLTR2 and an inhibitor of LTC4 biosynthesis.

The present invention further provides, in an aspect, a pharmaceutical composition comprising at least one antagonist of cysteinyl leukotriene receptor 2 (CysLTR2) for use in preventing or reducing an adverse side effect induced by a statin in muscle tissue of a subject, wherein the subject is receiving statin therapy and experiencing or at risk of experiencing an adverse side effect induced by the statin.

According to another aspect, the present invention provides a pharmaceutical composition comprising at least one inhibitor of LTC4 biosynthesis for use in preventing or reducing an adverse side effect induced by a statin in muscle tissue of a subject, wherein the subject is receiving statin therapy and experiencing or at risk of experiencing an adverse side effect induced by the statin, and wherein the at least one inhibitor of LTC4 biosynthesis inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP).

The present invention provides, in another aspect, a method for preventing or reducing an adverse side effect induced by a statin in muscle tissue of a subject, comprising the step of administering at least one antagonist of CysLTR2 to the subject, wherein the subject is receiving statin therapy and experiencing or at risk of experiencing an adverse side effect induced by the statin.

The present invention provides, in yet another aspect, a method for preventing or reducing an adverse side effect induced by a statin in muscle tissue of a subject, comprising the step of administering to the subject at least one inhibitor of LTC4 biosynthesis, wherein the subject is receiving statin therapy and experiencing or at risk of experiencing an adverse side effect induced by the statin, and wherein the inhibitor of LTC4 biosynthesis inhibits the activity of an enzyme selected from the group consisting of MGST2, cPLA2, 5-LO, and FLAP.

The field of cholesterol treatment is ever-growing and ever-changing, due to the extensive research and resources devoted to overcoming this condition. Therefore, as elaborated above, any type of molecule that affects cholesterol levels in humans is considered a “statin” according to the present invention. The term “statin” as used herein refers to any agent, molecule or drug used to lower cholesterol levels in humans by inhibiting the enzyme HMG-CoA reductase. In general, any molecule which reduces or eliminates the enzymatic activity of the enzyme HMG-CoA reductase is considered a “statin” by the present invention. Specifically, any molecule which attenuates HMG-CoA reductase from converting 3-hydroxy-3-methyl-glutaryl-CoA (HMG-CoA) to mevalonic acid is considered a “statin” by the present invention.

The term “pharmaceutical composition” as used herein refers to any composition comprising at least one biologically active agent, and at least one pharmaceutically acceptable carrier. Non-limiting examples of biologically active molecules are antagonists of leukotriene receptors, such as antagonists of the receptors CysLTR1 and CysLTR2, and inhibitors of LTC4 biosynthesis, such as inhibitors of the enzymes MGST2, cPLA2, 5-LO and FLAP. The term “agent” as used herein refers to any molecule having a biological activity.

As used herein, the term “pharmaceutically acceptable carrier” means a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. A carrier must be “acceptable” in the sense of being compatible with the other ingredients of the formulation and not injurious to the patient.

The terms “antagonist of a leukotriene C4 (LTC4) receptor”. “antagonist of CysLTR1”, “CysLTR1 antagonist”. “antagonist of CysLTR2”, and “CysLTR2 antagonist” are used herein to refer to any agent that is capable of blocking, inhibiting, reducing or interfering with the activity or function of the indicated leukotriene C4 receptor. These terms further refer to any agent that is capable of blocking, inhibiting, reducing or interfering with the expression of a leukotriene C4 receptor. Non-limiting examples of antagonists of a leukotriene C4 receptor are montelukast, zafirlukast and pranlukast.

As used herein, the term “inhibitor of leukotriene mediated activity” refers to leukotriene receptor antagonist, leukotriene biosynthesis inhibitor or a combination thereof. According to the findings of the present invention, both leukotriene receptor antagonists and leukotriene biosynthesis inhibitors are capable of inhibiting leukotriene mediated activity, and therefore may be used as alternative or in combination with each other. As used herein, the term “inhibitor of LTC4 mediated activity” refers to LTC4 receptor antagonist. LTC4 biosynthesis inhibitor or a combination thereof. As used herein, the term “leukotriene” refers to a leukotriene selected from the group consisting of: LTC4, LTD4, LTE4 and any combinations thereof. As used herein, the term “receptor antagonist” refers to a ligand of a receptor, which upon binding to the receptor exerts full or partial inhibition of the activity of that receptor, for example, LTC4 receptor antagonist causes inhibition of LTC4 receptor. As used herein, the term “ligand of a receptor” refers to a compound which specifically binds the receptor and thereby causes either activation or inhibition of the receptor. As used herein. “leukotriene receptor antagonist” is any compound capable of specifically binding to the leukotriene receptor, and capable of fully or partially inhibiting i.e. inactivating said receptor. The leukotriene receptor antagonist is thus a compound that exerts its prime effect though the binding and inactivation of the leukotriene receptor. The terms “LTC4 receptor antagonist”, “leukotriene receptor antagonist”, “CysLT1 receptor antagonist”, and “CysLT2 receptor antagonist” are intended here to cover any pharmaceutically acceptable salt, ester, solvate, or hydrate, which, upon administration to the recipient is capable of providing (directly or indirectly) the antagonist as described herein. The preparation of salts can be carried out by methods known in the art. According to some embodiments, the inhibitor of leukotriene mediated activity is selected from the group consisting of: inhibitor of leukotriene C₄ (LTC4) mediated activity, inhibitor of leukotriene D₄ (LTD₄) mediated activity, inhibitor of leukotriene E₄ (LTE₄) mediated activity, and any combinations thereof. Each possibility represents a separate embodiment of the present invention.

The term “preventing” generally refers to abrogating or delaying the initial onset of an acute and/or chronic adverse side effect associated with statin therapy in a subject receiving the statin. The prevention may be complete, e.g., total absence of damage to tissues or cells. The prevention may also be partial, such that damage to tissues or cells induced by statins is less than that which would have occurred without the effect of LTC4 receptor antagonist and/or LTC4 biosynthesis inhibitors.

The term “reducing” generally refers to attenuating the overall severity of and/or expediting the resolution of an acute and/or chronic adverse side effect associated with statin therapy in a subject receiving the statin.

The terms “treating” and “alleviating damage” interchangeably as used herein include the diminishment, alleviation, or amelioration of at least one symptom associated or induced by toxicity of the statin. The term “treating” as used herein also includes preventative (e.g., prophylactic), palliative and curative treatment.

The term “myopathy” as used herein refers to any one of the known muscular diseases, in which the muscle fibers do not function for any one of many reasons, resulting in muscular weakness. Muscle cramps, stiffness, and spasm can also be associated with myopathy. Muscular disease can be classified as neuromuscular or musculoskeletal in nature. Some conditions, such as myositis, can be considered both neuromuscular and musculoskeletal. Myopathies are either inherited or acquired. In certain embodiments, the term “myopathy” refers to a drug-induced myopathy, such as listed under International Classification of Diseases (ICD)-10-CM diagnosis code G72.0.

The term “myositis” as used herein generally refers to inflammation of the muscles. Elevation of creatine kinase (a marker of necrosis) in blood of subjects is usually indicative of myositis, as myositis often occurs as a result of cell necrosis. Types of myositis include, but are not limited to, myositis ossificans, idiopathic inflammatory myopathies, dermatomyositis, juvenile dermatomyositis, polymyositis, inclusion body myositis, and pyomyositis. In certain embodiments, the myositis is selected from the groups consisting of myositis ossificans and idiopathic inflammatory myopathies. Each possibility represents a separate embodiment of the invention.

The term “rhabdomyolysis” as used herein refers to a condition in which damaged skeletal muscle tissue breaks down rapidly. Breakdown products of damaged muscle cells are released into the bloodstream, some of these, such as the protein myoglobin, are harmful to the kidneys and may lead to kidney failure. The severity of the symptoms, which may include muscle pains, vomiting, and confusion, depends on the extent of muscle damage and whether kidney failure develops. The diagnosis is usually made with blood tests and urinalysis.

The terms “subject” or “a subject in need thereof” are used interchangeably herein and refer to a subject who is undergoing statin therapy and suffers from said adverse statin therapy side effects. According to some embodiment, the subject is a human. In some embodiments, the subject is a mammal.

In certain embodiments, the subject suffers from high levels of Low-density lipoprotein (LDL) cholesterol in the blood. In certain embodiments, the subject suffers from hypercholesterolemia. The terms “hypercholesterolemia”. “hypercholesterolaemia” and “dyslipidemia” as used herein refer to the presence of high levels of cholesterol in the blood. Hypercholesterolemia is a form of “hyperlipidemia” (elevated levels of lipids in the blood) and “hyperlipoproteinemia” (elevated levels of lipoproteins in the blood).

In certain embodiments, the subject receiving statin therapy is receiving high doses of statins. In certain embodiments, the phrase “receiving high doses of statins” means receiving at least 20, at least 30, at least 40, at least 50, at least 60, at least 80, or at least 80 mg of the statin daily.

The terms “comprise”, “comprises”. “comprising”. “include”, “includes”. “including”, “having” mean “including but not limited to”. The term “consisting of” means “including and limited to”. As used herein, the singular form “a”, “an” and “the” include plural references unless the context clearly dictates otherwise. For example, the term “a compound” or “at least one compound” may include a plurality of compounds, including mixtures thereof.

The term “inhibits the activity of a receptor” as used herein refers to any agent capable of preventing, blocking or attenuating the signal-transduction cascade or biological activity of a receptor. For example, cysteinyl leukotrienes activate CysLTR1 and CysLTR2, G protein-coupled receptors, to initiate a signal transduction cascade including the activation of MGST2 in non-hematopoietic cells.

The term “leukotriene biosynthesis inhibitor” as used herein refers to an inhibitor selected from the group consisting of: Microsomal glutathione S-transferase 2 (MGST2) inhibitor, cytosolic phospholipase A2 (cPLA2) inhibitor, 5-lipoxygenase (5-LO) inhibitor, 5-lipoxygenase Activating Protein (FLAP) inhibitor and combinations thereof. The terms “inhibitor of LTC4 biosynthesis” as used herein refers to any agent capable of preventing, blocking or attenuating the synthesis of LTC4. As detailed in the Background section, cPLA2 releases arachidonic acid from phospholipids, 5-LO and FLAP oxidize it to the reactive intermediate LTA₄ and LTC4 conjugates LTA₄ with glutathione to form LTC4. Any inhibitor of any one of these enzymes is considered an inhibitor of LTC4 biosynthesis. The term “inhibits the activity of an enzyme” as used herein refers to any agent capable of preventing, blocking or attenuating the activity of an enzyme.

The term “a therapeutically effective amount” as used herein refers to an amount of an agent which is effective, upon single or multiple dose administration to the subject, in providing a therapeutic benefit to the subject and/or in preventing an adverse side effect inflicted by a statin. In one embodiment, the therapeutic benefit is inhibiting or ameliorating symptoms of such adverse side effect. The term “a therapeutically effective amount” as used herein also refers to an amount of statin and/or inhibitor of leukotriene mediated activity which is effective, upon single or multiple dose administration to the subject, in providing a therapeutic benefit to the subject in need, such as, for example, cholesterol lowering effect, while attenuating the adverse statin therapy side effects.

As used herein, the term “administering” refers to bringing mammalian cells in contact with the compound or composition of the present invention. The effective amount of the composition used to practice the present invention for therapeutic treatment of conditions caused by or contributed to by the statins varies depending upon the statin, the regimen of the statin therapy, the manner of administration, the age, body weight, and general health of the patient. Ultimately, the attending physician will decide the appropriate amount and dosage regimen. Such an amount is referred to as an effective amount.

The terms “effective amount” and “amount effective” are used interchangeably herein to refer to the amount and/or dose of a composition comprising statin and/or inhibitor of leukotriene mediated activity upon single or multiple dose administration that is effective in attenuating the adverse statin therapy side effects. For example, in a non-limited manner, for treating a statin therapy side effect by the composition of the present invention, an effective amount of a statin is that which results in a measurable or detectable cholesterol lowering effect as that of the said statin when administered as a single therapy. According to some embodiments an effective amount of a leukotriene receptor antagonist is that which results in a measurable or detectable reduction, blocking, inhibition or prevention of the adverse effects of statin therapy.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The phrases “preventing or reducing” and “reducing or preventing” as used herein mean that a condition or a disease in a cell, a tissue or an organ of a subject is prevented or reduced in severity by the administration of an agent prior to, during, or after the appearance of the condition or the disease, or a symptom thereof. Therefore, in some embodiments, the phrases “preventing or reducing” and “reducing or preventing” mean “preventing”. In other embodiments, the phrases “preventing or reducing” and “reducing or preventing” mean “reducing”.

In certain embodiments, the statin is selected from the group consisting of atorvastatin, fluvastatin, lovastatin, pitavastatin, pravastatin, rosuvastatin, and simvastatin. Each possibility represents a separate embodiment of the invention.

According to some embodiments, the CysLTR2 antagonist is selected from the group consisting of: BAY-cysLT2 (CAS Number 712313-33-2), BAY-u9773 (CAS Number 154978-38-8). HAMI3379 (CAS Number 712313-35-4), and any combination thereof. Each possibility represents a separate embodiment of the present invention.

In certain embodiments, the antagonist of CysLTR2 is dual-specific and further inhibits the activity of CysLTR1. In certain embodiments, the pharmaceutical compositions described above comprise a mixture or a plurality of LTC4 receptor antagonists, comprising an antagonist of CysLTR1 and an antagonist of CysLTR2.

Besides interfering or blocking the interaction between LTC4 and its receptors, CysLTR1 and CysLTR2, LTC4's biological activity may be minimized by abrogating LTC4 biosynthesis in the subject's body. In certain embodiments, the pharmaceutical compositions described above comprise an antagonist of CysLTR2. In certain embodiments, the pharmaceutical compositions described above comprise an inhibitor of LTC4 biosynthesis which inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP). Each possibility represents a separate embodiment of the invention. In certain embodiments, the inhibitor of LTC4 biosynthesis inhibits the activity of MGST2. In certain embodiments, the inhibitor of LTC4 biosynthesis inhibits the activity of cPLA2. In certain embodiments, the inhibitor of LTC4 biosynthesis inhibits the activity of 5-LO. In certain embodiments, the inhibitor of 5-LO is zileuton. In certain embodiments, the inhibitor of 5-LO is atreleuton. In certain embodiments, the inhibitor of LTC4 biosynthesis inhibits the activity of FLAP. In certain embodiments, the inhibitor of FLAP is MK-886.

In certain embodiments, the pharmaceutical compositions described above comprise a statin, an antagonist of CysLTR2, and an inhibitor of LTC4 biosynthesis which inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP).

In certain embodiments, the pharmaceutical compositions described above are for use in preventing or reducing an adverse side effect induced by a statin in muscle tissue of a subject, wherein the subject is receiving statin therapy and experiencing or at risk of experiencing an adverse side effect induced by the statin.

According to certain embodiments, the adverse side effect is a statin-induced myopathy. In certain embodiments, the myopathy is selected from the group consisting of myositis and rhabdomyolysis. Each possibility represents a separate embodiment of the invention.

In certain embodiments, the subject is receiving at least 20 mg of atorvastatin daily or 40-80 mg of atorvastatin daily. In certain embodiments, the subject is receiving 0.8 mg of cerivastatin daily. In certain embodiments, the subject is receiving more than 80 mg of fluvastatin daily. In certain embodiments, the subject is receiving more than 80 mg of lovastatin daily. In certain embodiments, the subject is receiving at least 4 mg of pitavastatin daily. In certain embodiments, the subject is receiving more than 40 mg of pravastatin daily. In certain embodiments, the subject is receiving at least 10 mg of rosuvastatin daily or 20-40 mg of rosuvastatin daily. In certain embodiments, the subject is receiving at least 40 mg of simvastatin daily or 80 mg of simvastatin daily. Each possibility represents a separate embodiment of the invention.

Patients suffering from high levels of cholesterol are often being treated by a statin, or a combination of statins. Since these statins are administered systemically, they often inflict adverse side effect, in part by interacting with muscle cells. In certain embodiments, the statin treats the high levels of cholesterol. In certain embodiments, the statin induces the adverse side effect.

In certain embodiments, the kit described above comprises a pharmaceutical composition comprising a statin, a pharmaceutical composition comprising an antagonist of CysLTR2, and a pharmaceutical composition comprising an inhibitor of LTC4 biosynthesis. In certain embodiments, the kits described above further comprise instructions for administering the statin and the agent to a subject receiving statin therapy and experiencing or at risk of experiencing an adverse side effect induced by the statin. In certain embodiments, the kits described above are for use in preventing or reducing an adverse side effect induced by a statin in muscle tissue of a subject, wherein the subject is receiving statin therapy and experiencing or at risk of experiencing an adverse side effect induced by the statin. In certain embodiments, the use comprises administering the agent prior to, during, and/or after administering the statin to the subject. Each possibility represents a separate embodiment of the invention.

According to some embodiments, the pharmaceutical compositions of the present invention further comprise an effective amount of a statin selected from the group consisting of: atorvastatin, cerivastatin, fluvastatin, lovastatin, mevastatin, pitavastatin, pravastatin, rosuvastatin, simvastatin, and any combination thereof. Each possibility represents a separate embodiment of the present invention.

According to some embodiments, use of combination therapy by the pharmaceutical composition of the present invention will provide similar or better cholesterol lowering effect as that of the statin when administered as a single therapy, while reducing its side effects.

According to additional embodiments, the dosages used for statin is in a range similar to that of the approved drugs when given separately for their approved indications.

Statin therapy is a category of cholesterol treatment. To prevent or reduce adverse side effects inflicted by statins, such as myopathies, it must be understood that the pharmaceutical compositions described above, comprising an antagonist of a LTC4 receptor, an inhibitor of LTC4 biosynthesis, or any combination thereof, may be administered to the subject being treated by a statin, prior to the treatment by the statin, at the same time with the treatment by the statin, together with the treatment by the statin, after the treatment by the statin, and in any combination thereof. Each possibility represents a separate embodiment of the invention.

In certain embodiments, the subject has not been previously treated by the statin. In other certain embodiments, the subject has been previously treated by a statin similar in function to the statin. In certain embodiments, the subject has been previously treated by the statin. In certain such embodiments, the subject has been previously treated by the statin, and is known to obtain an adverse side effect inflicted by the statin.

Any suitable route of administration to a subject may be used for the composition of the present invention. The preferred mode of administration will depend upon the particular indication being treated and will be apparent to one of skill in the art.

According to some embodiments, the pharmaceutical compositions of the present invention may be provided as a pill, tablet or capsule, for oral administration.

According to some embodiments, the pharmaceutical compositions of the present invention may be provided as a powder, solution or suspension, for intravenous administration.

In certain embodiments, the pharmaceutical compositions provided by the present invention are formulated for the extended release of the statin. The term “extended release” in respect to the formulations disclosed herein means that the formulation does not immediately release the active ingredient to the environment (e.g., blood, stomach, intestine, colon), but rather releases the active ingredient over a predetermined amount of time. Thus, relatively constant or predictably varying amounts of the active agent can be delivered over a specified period of time. Expressions such as “prolonged action,” “repeat-action.” “sustained release”, “modified release” and “controlled release” have also been used to describe such formulations or dosage forms. An extended release can therefore be described as a dosage form or a formulation that allows at least a two-fold reduction in dosing frequency as compared to a conventional immediate release dosage form. As used herein, the term “active ingredient” means a statin, an antagonist of cysLTR2 and/or an inhibitor of LTC4 biosynthesis.

In certain embodiments, the pharmaceutical compositions provided by the present invention comprise 10, 20, 30, 40, 50, 60, 70 or 80 mg of the statin.

The combination of components constituting the treatment may be administered either simultaneously (as discrete dosage forms or as a single composition), sequentially, or separated by a suitable time interval. Each possibility represents a separate embodiment of the present invention. Where the components are administered as discrete dosage forms, i.e., not as combined compositions, each component may be administered in the same form or a different form, e.g., oral, nasal, parenteral, or dermal. When the compounds are administered simultaneously, sequentially or separately, the components may be provided as discrete dosage forms.

In certain embodiments, the present invention provides a combination therapy of a statin selected from a group containing atorvastatin, cerivastatin, fluvastatin, lovastatin, mevastatin, pitavastatin, pravastatin, rosuvastatin, simvastatin or any other inhibitor of cholesterol biosynthesis, together with an inhibitor of LTC4 activity selected from a group containing approved drug such as zileuton and experimental drugs such as BAY-cysLT2, MK-886, atreleuton or the like. The dosage of the combination therapy will be in a range similar to that of the approved drugs when given separately. The combination therapy may be given by any approved administration form and is preferably provided as single pills containing both statin and an inhibitor of the MGST2-LTC4 pathway for oral administration.

In one aspect, the LTC4 inhibitors are RNA interference agents capable of inhibiting the expression of polypeptides selected from the group consisting of: LTC4, MGST2, cPLA2, 5-LO, FLAP. CysLTR2 and any combinations thereof. Each possibility represents a separate embodiment of the present invention.

According to the present invention, an “RNA interference agent” or “RNAi agent” is either a double stranded RNA or a DNA construct engineered to be capable of transcribing a double stranded RNA within a target cell, which double stranded RNA comprises an RNA strand which is at least 70% complementary to the nucleotide sequence of a polypeptide selected from the group consisting of: LTC4. MGST2, cPLA2, 5-LO, FLAP, CysLTR2 or to a homolog or fragment or portion thereof.

According to further embodiment, the present invention provides an antisense oligonucleotide sequence complementary to the nucleotide sequence of a polypeptide selected from the group consisting of LTC4, MGST2, cPLA2, 5-LO, FLAP, CysLTR2 or a homolog or fragment thereof, wherein the antisense oligonucleotide sequence is capable of specifically hybridizing with said polypeptide or a homolog or fragment thereof, thereby inhibiting expression of LTC4.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1— ER Stress Regulates Expression of MGST2 and 5-LO

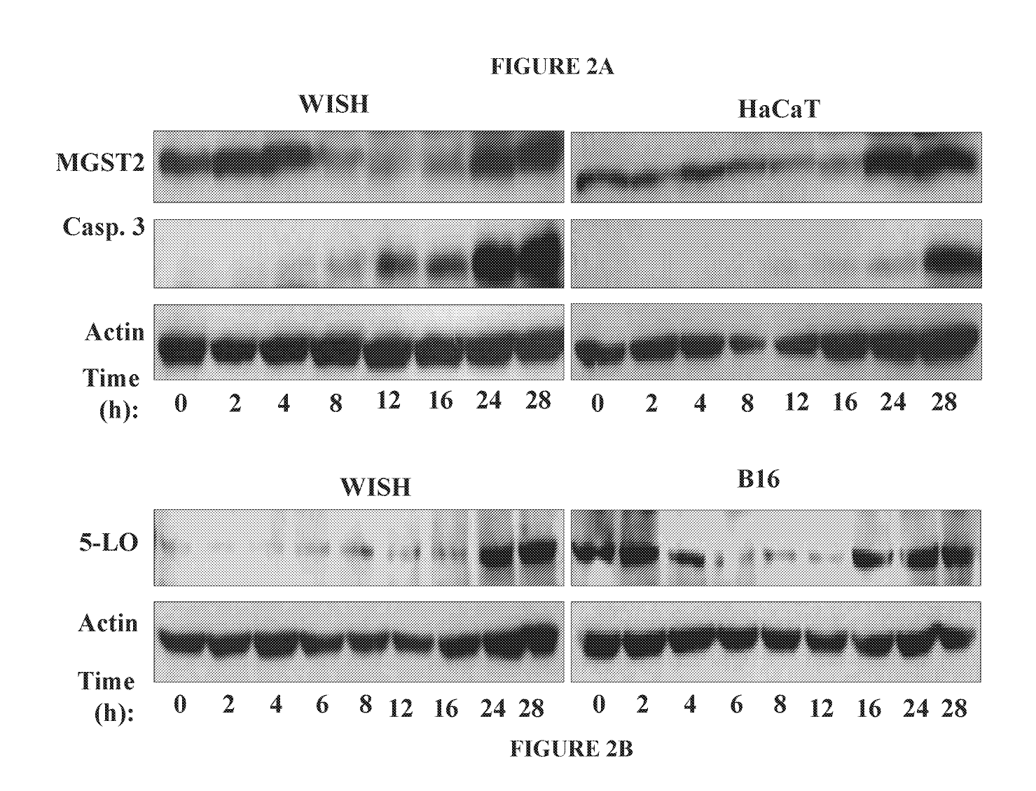

To determine the impact of ER stress on the levels of MGST2 in human WISH epithelial cells and human undifferentiated HaCaT keratinocytes, kinetics of microsomal glutathione S transferase (MGST2) and cleaved caspase 3 (Casp. 3) expression following treatment of human WISH epithelial cells and human HaCaT pre-keratinocytes with tunicamycin, a protein glycosylation inhibitor (1 μg/ml, 24 hours), was determined by immunoblotting. The results shown in FIG. 2A demonstrate that the treatment of these cells with the tunicamycin leads to ER stress, presumingly due to protein misfolding. Remarkably, in both cell types. MGST2 was down regulated during the early, protective phase of the unfolded protein response (UPR), and up regulated at the late, death promoting phase, concomitantly with elevation of cleaved caspase 3. To assess the role of MGST2 and its product LTC4 in ER stress-triggered cell death, an immunoblotting test was performed for determining the kinetics of 5-lipoxygenase (5-LO) expression following treatment of human WISH epithelial cells with Brefeldin A, an inhibitor of protein transport from the endoplasmic reticulum (ER) to the Golgi apparatus (0.66 μg/ml, 24 hours), and mouse B16 cells with tunicamycin (1 μg/ml, 24 hours). The results shown in FIG. 2B demonstrate that 5-LO, the rate-limiting enzyme of leukotriene biosynthesis, also exhibited an inverse bell-shaped expression pattern in response to ER stress. Hence, the two key components of LTC4 biosynthesis are tightly regulated by ER stress. They are attenuated at the early, pro-survival phase of the UPR, and are up regulated at the late, death-promoting phase.

### Example 2—ER Stress Triggers Nuclear Translocation of MGST2 and 5-LO

To evaluate the impact of ER stress on the cellular localization of MGST2, cPLA2, 5-LO and FLAP, immunostaining of WISH epithelial cells untreated and treated with Brefeldin A (0.66 μg/ml, 24 hours), was performed. The results show that that in control human WISH cells MGST2 was present both in the ER and in the nucleus of the cells (data not shown) and that 5-LO was present both in the cytoplasm and in the nucleus of the cells (data not shown). Despite their common nuclear location in control cells, 5-LO did not co-localize with MGST2 either in human WISH epithelial cell or HaCaT pre-keratinocytes (data not shown). Triggering ER stress with Brefeldin A, a toxin that blocks the passage of proteins from the ER to the Golgi apparatus, resulted in translocation to the nucleus of both MGST2 and 5-LO, as determined by staining with the nuclear envelope marker lamin and the nuclear marker Hoechst 33258 (data not shown). Furthermore, ER stress triggered co-localization of MGST2 and 5-LO in both WISH and HaCaT cells (data not shown).

### Example 3—ER Stress Triggers Nuclear Translocation of the Entire Biosynthetic Machinery of LTC4

Activation of Fc receptor in mast cells initiated LTC4 production by triggering translocation of its entire biosynthetic machinery to the nuclear envelope. Therefore, in order examine the impact of ER stress on the cellular localization of the two other enzymes required for biosynthesis of LTC4, translocation and co-localization of 5-LO activating protein (FLAP) and 5-LO in WISH cells following treatment with Brefeldin A (0.66 pig/ml, 24 hours), was determined by immunostaining. The results show that in control cells FLAP was localized in the nucleus and the ER whereas cPLA2 was mainly in the cytoplasm. Despite their overlapping locations in control cells, 5-LO did not co-localize with FLAP or cPLA2 (data not shown). Triggering ER stress with Brefeldin A elicited translocation to the nucleus and co-localization of 5-LO with FLAP and cPLA2 (data not shown). Thus. ER stress triggers the translocation to the nucleus and co-localization of a set of enzymes capable of generating LTC4. This ER stress-triggered translocation event is analogous to the one seen upon Fc receptor activation of mast cells, except that it is induced by ER stress rather than an immunological cue and it involves MGST2 rather than LTC4S.

### Example 4—ER Stress Triggers MGST2-Based Biosynthesis of LTC4

To further evaluate whether the ER stress-triggered translocation of MGST2-based machinery resulted in production of LTC4, an immunostaining of EDC-fixed cells was performed to determine the impact of three inducers of ER stress, which act by different mechanisms, on LTC4 biosynthesis was performed. Tunicamycin inhibits protein glycosylation, thapsigargin triggers release of calcium ions from the ER to the cytoplasm and Brefeldin A blocks protein transport from the ER to the Golgi. The results demonstrate that induction of ER stress by these toxins elicited an extensive biosynthesis of LTC4. Altogether, these results suggest that ER stress triggers MGST2-mediated LTC4 biosynthesis by expression, nuclear translocation and co-localization of MGST2, 5-LO, FLAP and cPLA2.

### Example 5—ER Stress Regulates Expression and Translocation of the LTC4 Receptors to the Nucleus

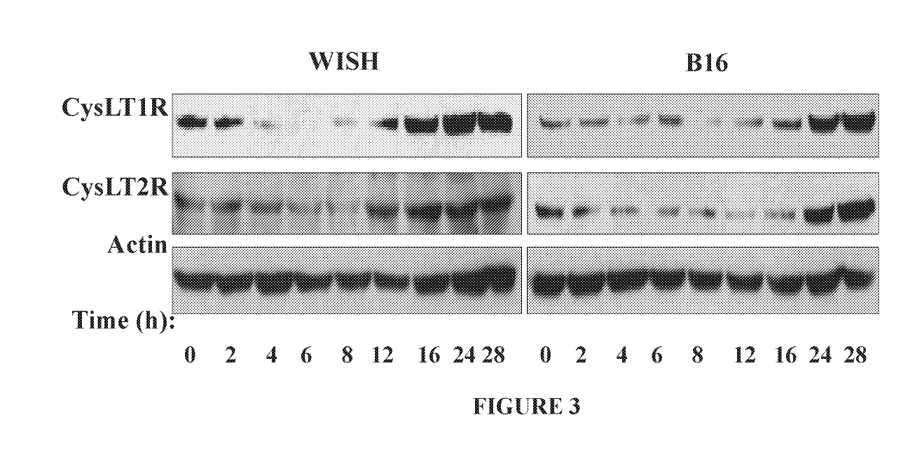

In mast cells, LTC4 is secreted and acts on adjacent cells by binding to its two cytoplasmic membrane receptors. CysLTR1 and CysLTR2. However, it was previously demonstrated that exogenously added LTC4 and LTD4 trigger translocation of their receptors to the nuclear envelope (Nielsen. C. K., et al. Cancer Res, 2005. 65(3): p. 732-42). Kinetics analysis of CysLTR1 and CysLTR2 expression following treatment of human WISH cells with Brefeldin A (0.66 μg/ml, 24 hours), and mouse B16 cells with tunicamycin (1 μg/ml, 24 hours), was performed by using immunoblotting. Surprisingly, as can be seen from the results shown in FIG. 3, in both human WISH epithelial cells and mouse B16 cells CysLTR1 and CysLTR2 were down regulated during the early, protective phase of the UPR and restored and further induced at the late death-promoting phase of the UPR.

In addition, the results demonstrate the unexpected and surprising finding, that ER stress also triggered translocation of CysLTR1 and CysLTR2 to the nuclear envelope and their co-localization with 5-LO. Thus, following ER stress, both the LTC4 biosynthetic machinery and the LTC4 receptors are co-localized in the same cellular compartment, thereby facilitating intracrine action of the MGST2-generated LTC4.

### Example 6—ER Stress Triggers MGST2-Based Biosynthesis of LTC4

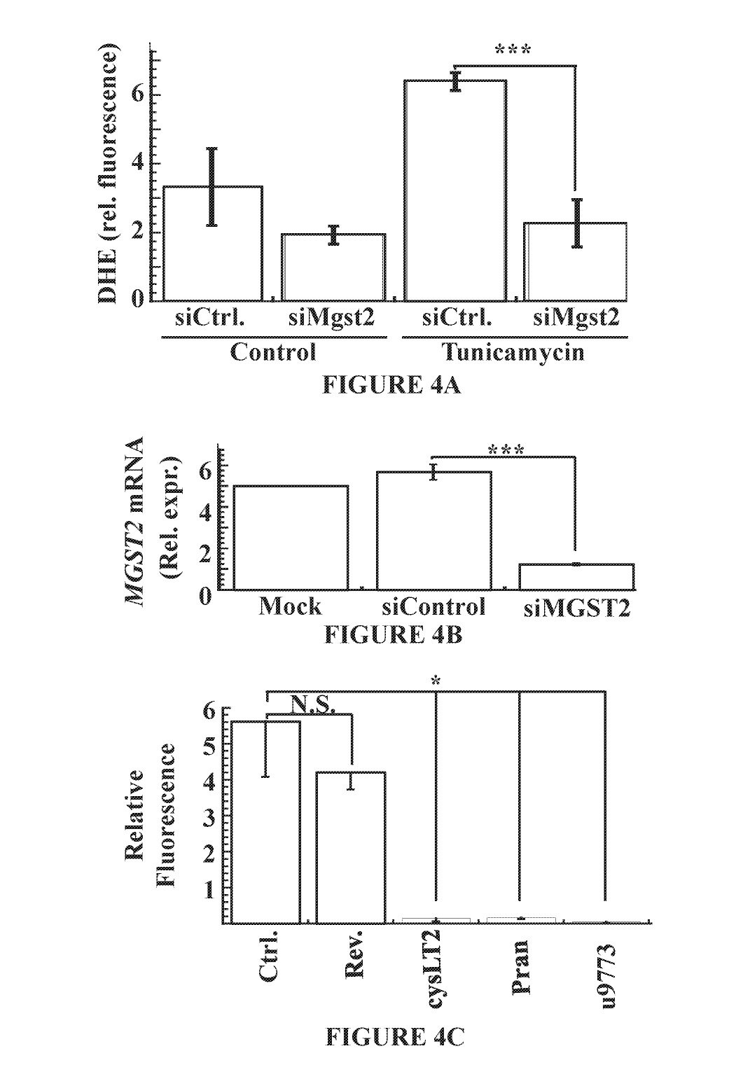

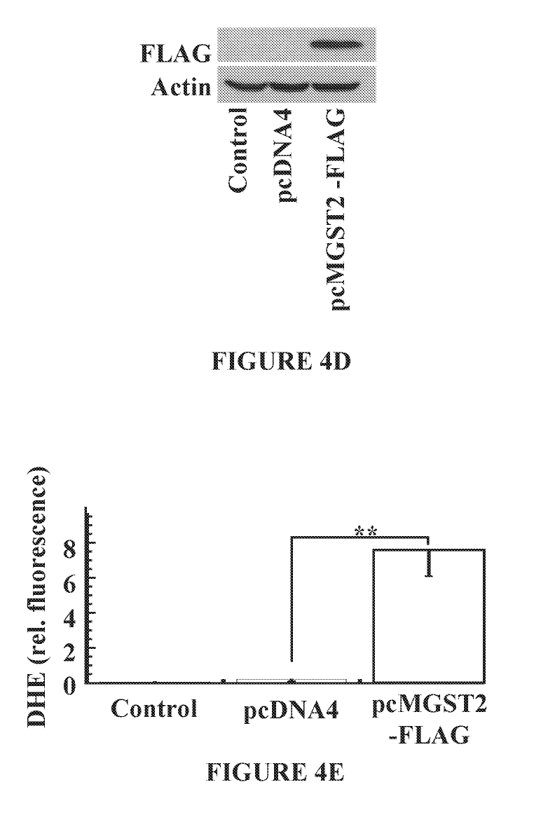

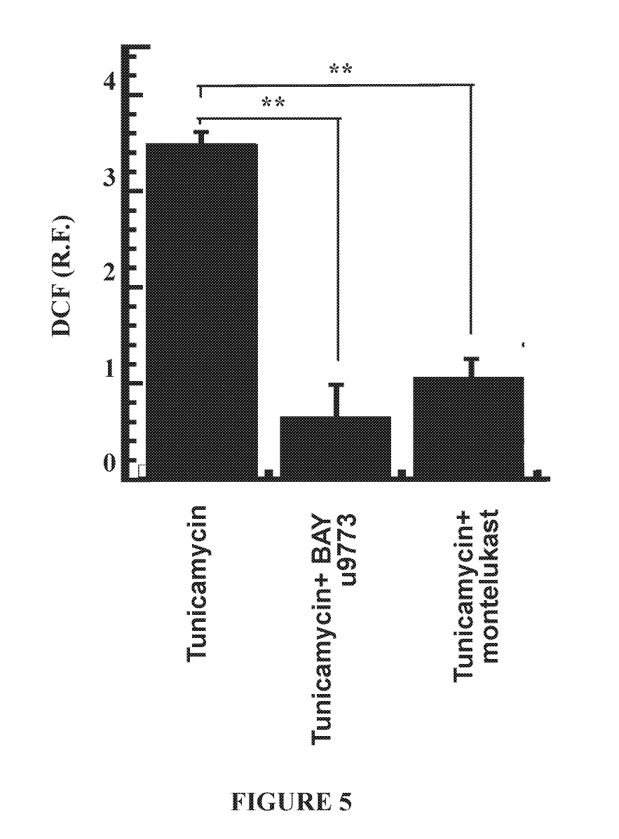

ER stress and oxidative stress are tightly associated, as ER stress triggers oxidative stress and vice versa. Therefore, a test for determining whether MGST2 and its product LTC4 are involved in ER stress-triggered ROS accumulation was performed. As previously reported, ER stress triggered by tunicamycin leads to accumulation of ROS, as detected by staining with the superoxide anion indicator dihydroethidium (DHE). Upon effective silencing of MGST2 with specific siRNA (FIG. 4B), the level of ER stress triggered ROS accumulation was significantly reduced, reaching that of control cells (FIG. 4A). MGST2 consumes GSH by coupling it not only to LTA₄ but also to other substrates. To find out if MGST2 triggers ROS accumulation by depletion of GSH or by biosynthesis of LTC4, several LTC4 receptor antagonists as well as inhibitors of LTC4 biosynthesis or transport were employed. Because leukotrienes mediate the symptoms of asthma, many different inhibitors of leukotriene biosynthesis, transport and activity are available. Reversan inhibits LTC4 export from the cell. Montelukast and pranlukast are selective CysLTR1 receptor antagonists, BAY-cysLT2 is a selective CysLTR2 antagonist and BAY-u9773 is a dual inhibitor of both CysLTR1 and CysLTR2. Triggering ER stress by Brefeldin A generated ROS, as measured by the indicator dichloro-dihydro-fluorescein diacetate (DCFH-DA, FIGS. 4C, 5). The results revealed surprisingly and unexpectedly that all CysLTR1 & CysLTR2 antagonists tested significantly inhibited ER stress-triggered ROS accumulation (FIGS. 4C, 5).

In contrast with the LTC4 antagonists, the MRP1 transporter inhibitor reversan (20 μM) had a small but statistically insignificant inhibitory effect on ROS accumulation in response to ER stress (FIG. 4C), indicating that LTC4 activity is mostly intracrine. This important finding was in line with the previously findings that the two LTC4 receptors translocate to the nucleus, further supporting the notion that ER stress-triggered LTC4 action is mainly intracrine. The role of MGST2 in ROS accumulation was further established by its over expression in HEK 293T cells (FIG. 4D), which resulted in a marked elevation in cellular ROS (FIG. 4E). Taken together, these results indicate that MGST2-generated LTC4 is the major mediator of ER stress triggered oxidative stress.

### Example 7—the MGST2-LTC4 Pathway Activates Nuclear NADPH Oxidase 4 (NOX4) and Subsequent DNA Damage

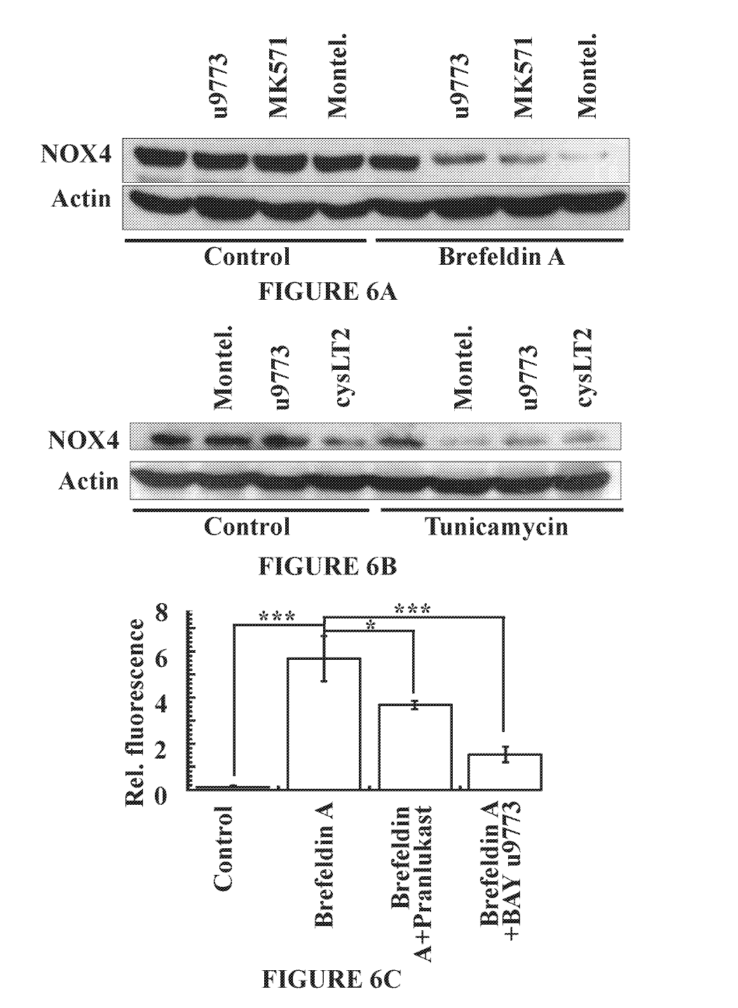

Recent studies have identified NADH/NADPH oxidases (NOX) as the most important cellular ROS producing enzymes and ER stress was found to trigger oxidative stress by up-regulating the NOX4 isoform. Several ligands of G-protein coupled receptors to which CysLTR1 and CysLTR2 belong elicit oxidative stress through receptor-mediated activation of NOX. Human WISH cells and Mouse B16 cells were treated with vehicle (Control) or Brefeldin A (0.66 μg/ml, 24 hours) in the absence or presence of BAY-u9773, MK571 or Montelukast and NOX4 levels were evaluated by immunoblotting. Remarkably, LTC4 receptor antagonists greatly inhibited NOX4 expression upon ER stress (FIGS. 6A, 6B). Furthermore, treating WISH cells with vehicle (Control) or Brefeldin A (0.66 μg/ml, 24 hours), fixed and immunostained revealed that ER stress triggered translocation of NOX4 towards the nucleus. Hence, expression and activation of NOX4, the major ROS producer under ER stress, is LTC4-dependent.

Accumulation of nuclear ROS in response to ER stress will result in oxidative DNA damage. To test whether the MGST2-LTC4 pathway is involved in ER stress-triggered DNA damage, the formation of the oxidized DNA derivative 8-hydroxy-2′dexoxyguanosine (8-OHdG) was measured. Treatment of HaCaT cells with Brefeldin A resulted in appearance of 8-OHdG at the nucleus, as determined by immunostaining (FIG. 6C). Inhibition of LTC4 binding to CysLTR1 with pranlukast significantly attenuated the formation of nuclear 8-OHdG and inhibition of LTC4 binding to both CysLTR1 and CysLTR2 using BAY-u9773 was even more effective in attenuating the DNA damage (FIG. 6C). Altogether, the ER stress-triggered DNA damage was mediated to a very large extent by the MGST2-LTC4 pathway.

### Example 8—the MGST2-LTC4 Pathway Mediates ER Stress Triggered Cell Death

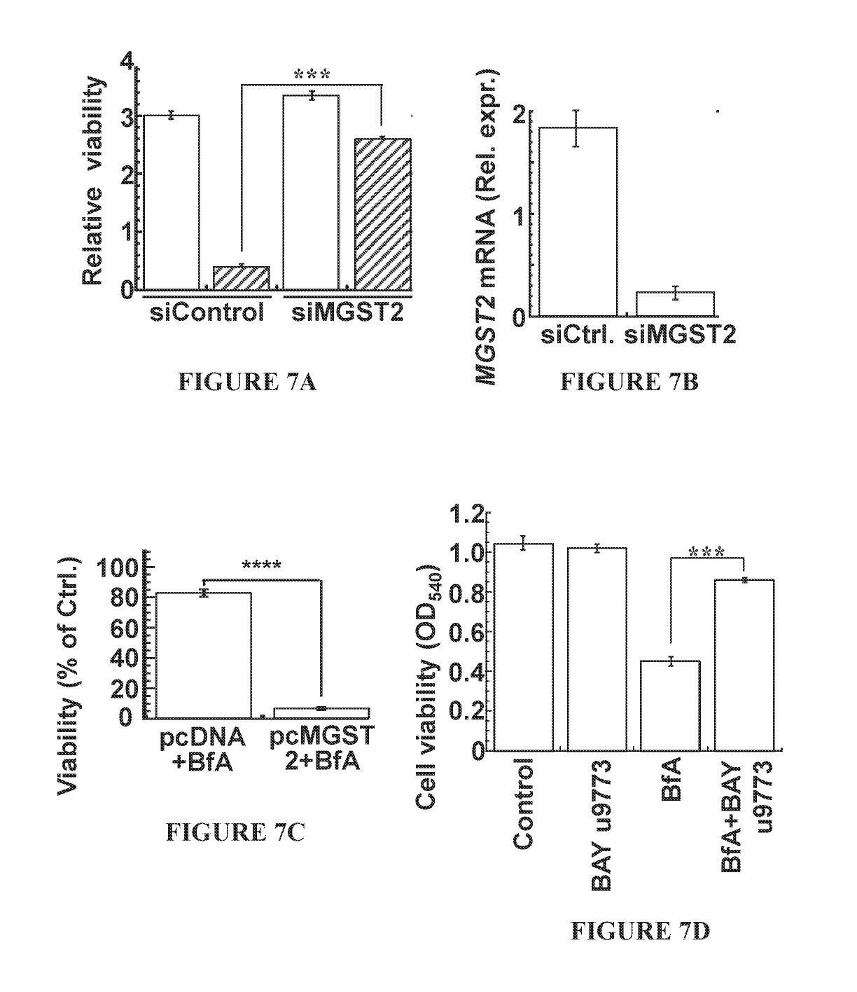

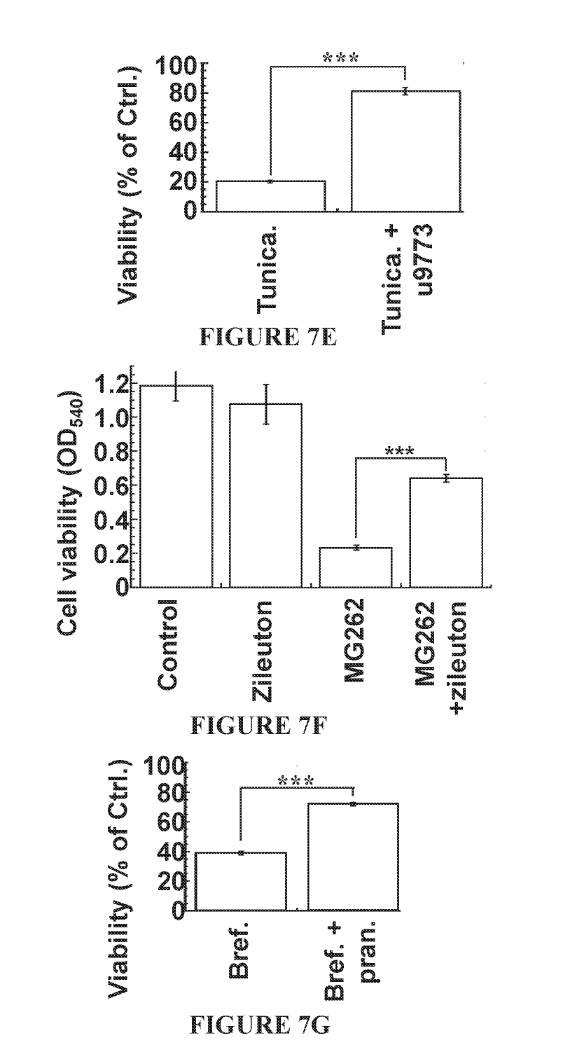

Although moderate levels of ROS promote cell proliferation, concentrations above a level termed “the toxic threshold”, trigger cell death. If LTC4 elevates ROS above this threshold, then it may be part of the ER stress-triggered cell death machinery, as reported in the case of cardiomyocytes death by hypoxia/reperfusion. Furthermore, induction of NOX2 was identified as one of the mechanisms leading to ER stress-triggered apoptosis of macrophages. To determine whether MGST2-LTC4 Pathway Mediates ER Stress Triggered Cell Death, mouse B16 cells were transfected with control siRNA (siControl) or MGST2-specific siRNA (siMGST2), treated with vehicle or tunicamycin (1 μg/ml, 24 hours) (Tm) and then stained with Crystal violet. The results demonstrate that effective knockdown of MGST2 significantly attenuated ER stress triggered death of B16 melanoma cells (FIGS. 7A, 7B). In an inverse study, over-expression of MGST2 in HEK 293T cells significantly augmented cell death (FIG. 7C). To conclude, MGST2 is involved in ER stress triggered cell death.

Next, another test to examine the role of the MGST2 product LTC4 in ER stress triggered cell in Human HaCaT pre-keratinocytes was performed. HaCaT cells were treated with vehicle (Control) or Brefeldin A (BfA, 1.33 μg/ml, 48 hours) in the absence or presence of BAY-u9773 (80 nM). and then stained with Crystal violet.

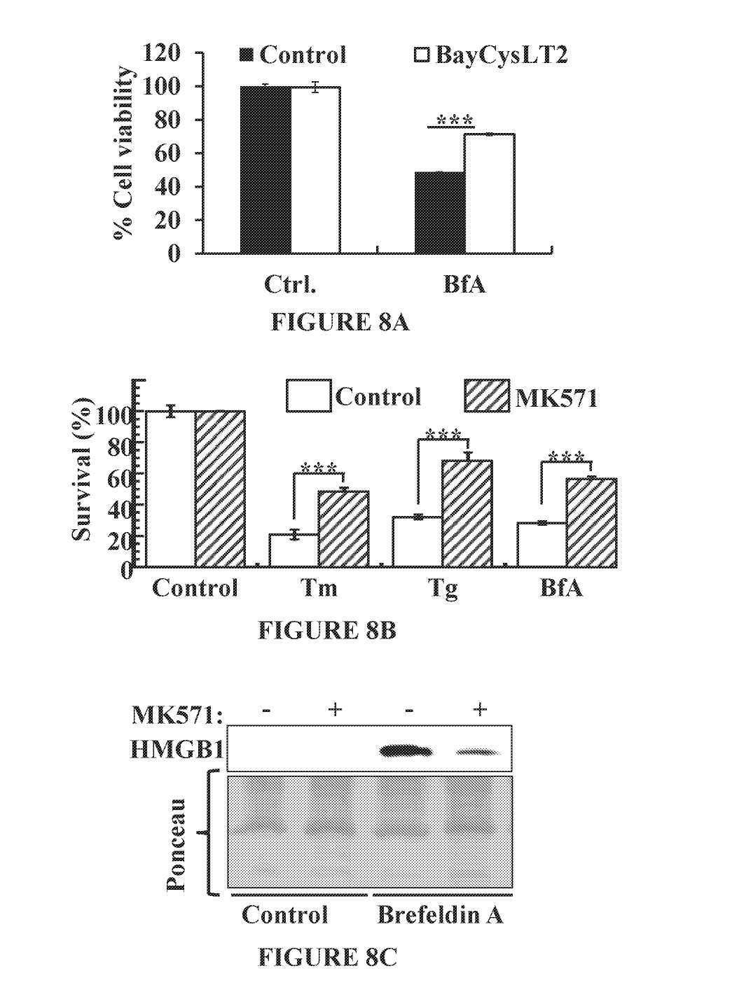

The results show that Pre-treatment of human HaCaT keratinocytes with the dual CysLTR1 and CysLTR2 antagonist, BAY-u9773 attenuated ER stress triggered cell death elicited by Brefeldin A (FIG. 7D). BAY-u9773 also significantly attenuated tunicamycin-induced death of human WISH cells as determined by Neutral red assay (FIG. 7E). Proteasome inhibitors trigger ER stress and subsequent cell death due to accumulation of misfolded proteins in the ER. The 5-lipoxygenase inhibitor zileuton was previously found to reduce apoptotic cell death following spinal cord injury and cerebral ischemia. The results show that zileuton (10 μM) significantly protected HaCaT keratinocytes from death triggered by the proteasome inhibitor MG262 (50 nM, 24 hours, FIG. 7F). The two LTC4 receptors CysLTR1 and CysLTR2 dimerize in mast cells and in human intestinal epithelial cells. Hence it is likely that selective inhibition of one of these receptors would be sufficient for attenuating LTC4 activity. Indeed, pranlukast (10 μM), a selective CysLTR1 antagonist, significantly attenuated Brefeldin A-triggered death of human WISH cells (FIG. 7G). Similarly. BAY-cysLT2 (5 μM), a selective CysLTR2 antagonist, equally protected these cells from Brefeldin A (FIG. 8A).

On top of its activity as an MRP1 inhibitor, MK571 is a potent selective CysLTR1 antagonist. Pre-treatment of murine B16 melanoma cells with MK571 (10 μM) significantly attenuated cell death triggered by tunicamycin, thapsigargin and Brefeldin A (FIG. 8B). This protective effect correlated with reduction of cellular necrosis, as demonstrated by reduced secretion of HMGB1 to the culture medium (FIG. 8C).

All together, the results provided herein identified a previously unrecognized ER stress-triggered signaling pathway that mediates ER stress triggered cell death.

### Example 9—MGST2 Deficiency Attenuates ER Stress-Triggered Death In Vivo

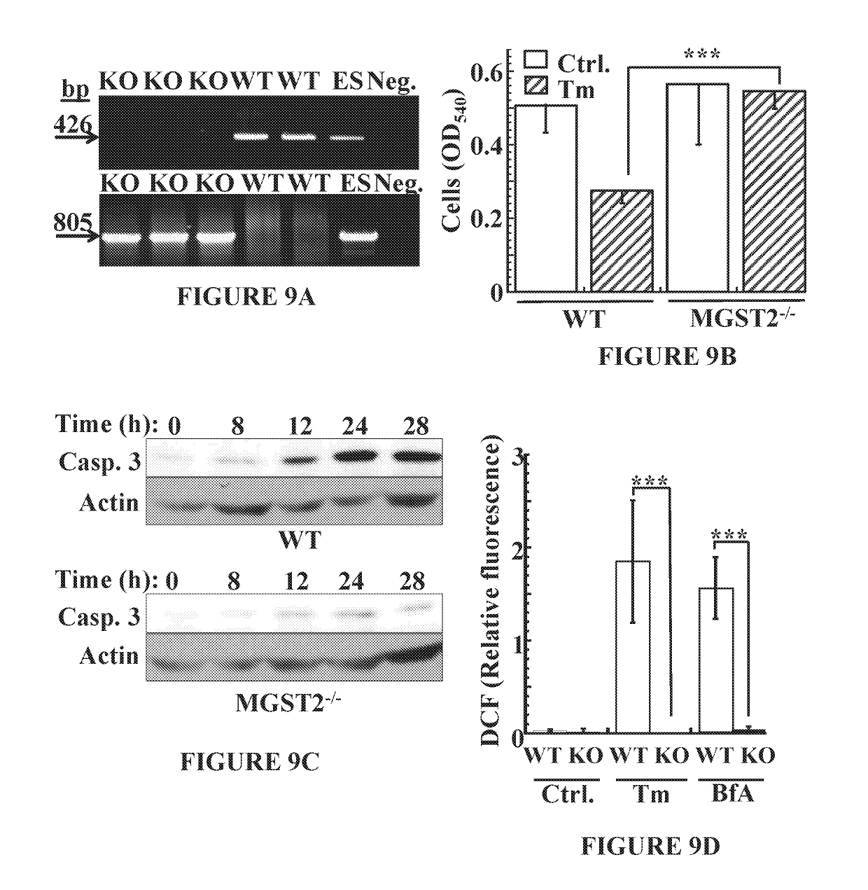

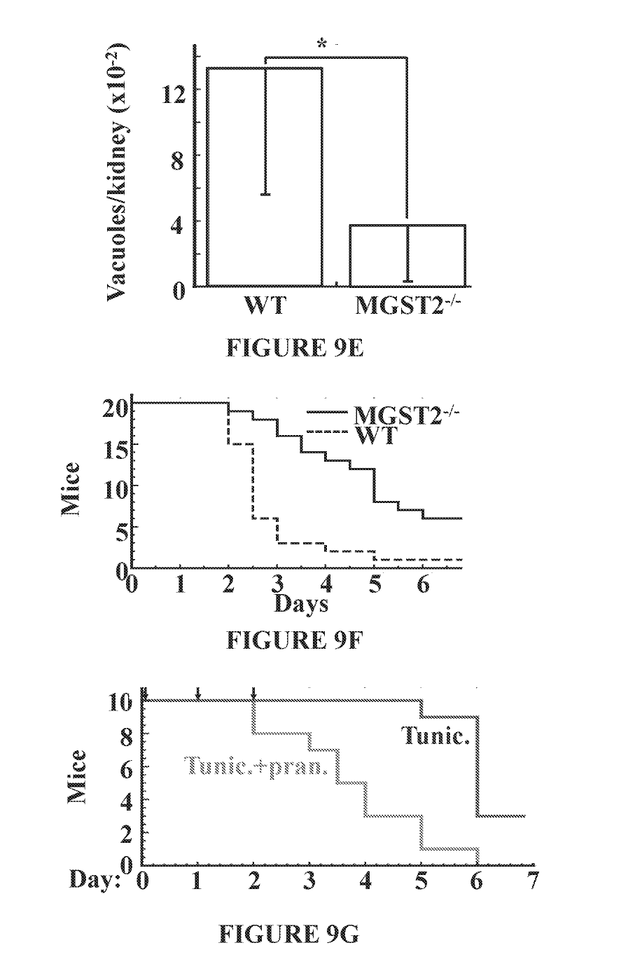

Homozygous MGST2 deficient mice were established from the 129svEvBrd mouse strain gene trap library of ES cells. PCR of DNA samples obtained from tail tips of Mgst2 knockout mice and their WT and heterozygous littermates confirmed that the targeted allele of Mgst2 is a null mutation (FIG. 9A). The MGST2-deficient mice bred normally and appeared indistinguishable from their wild-type littermates, with no significant differences in body weight and food intake. Fibroblasts of WT and MGST2-deficient mouse embryos at passage 2 were treated with vehicle (Control) or tunicamycin (1 μg/ml, 24 hours). The plates were then stained with Crystal violet. The results show that MGST2-deficient murine embryonic fibroblasts (MEFS) at passage 2 were significantly more resistant than WT MEFS to ER stress-triggered cell death, elicited by tunicamycin (FIG. 9B). Staining with DCFH-DA revealed practically complete lack of ROS accumulation in response to ER stress, as compared with WT MEFs (FIG. 9C).

To evaluate the role of MGST2 in ER stress in vivo, a mouse model of acute kidney injury was employed. WT and MGST2-deficient mice were injected ip at day 0 with tunicamycin (1 mg/kg body weight). Histological examination of hematoxylin-eosin stained WT kidney sections, collected at day 4, revealed the expected damage to the proximal tubules of WT mice, whereas the histological picture in the MGST2-deficient mice was significantly milder, as determined by the number of vacuoles (FIGS. 9D, 9E). To further study the role of the MGST2-LTC4 pathway in ER stress triggered morbidity, a higher dose of tunicamycin was injected to mice. MGST2 deficient mice were dramatically more resistant than WT mice to a single tunicamycin dose of 2.5 mg/kg (FIG. 9F). Furthermore, administration of pranlukast concomitantly with 2.5 mg/kg tunicamycin greatly reduced the morbidity and mortality of WT mice as compared with tunicamycin alone (FIG. 9G).

### Example 10—Pranlukast Protects Human WISH Cells from Simvastatin-Triggered Cell Death

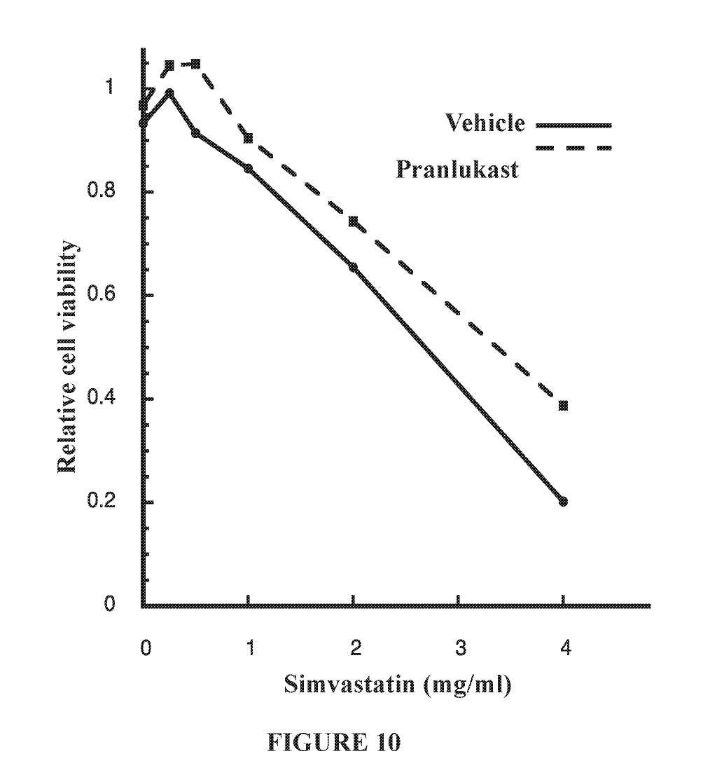

To study the role of the MGST2-LTC4 pathway in statin-triggered cell death human WISH epithelial cells in 96 well plates were treated with various concentrations of simvastatin or vehicle in the presence of either pranlukast (10 μM) or vehicle for 48 h. The results show that pranlukast attenuated cell death triggered by a broad range (0.5-4 μg/ml) of simvastatin concentrations (FIG. 10).

### Example 11—BAY-cysLT2 and BAY-u9773 Protect Mouse C2C12 Myocyces from Simvastatin-Triggered Cell Death

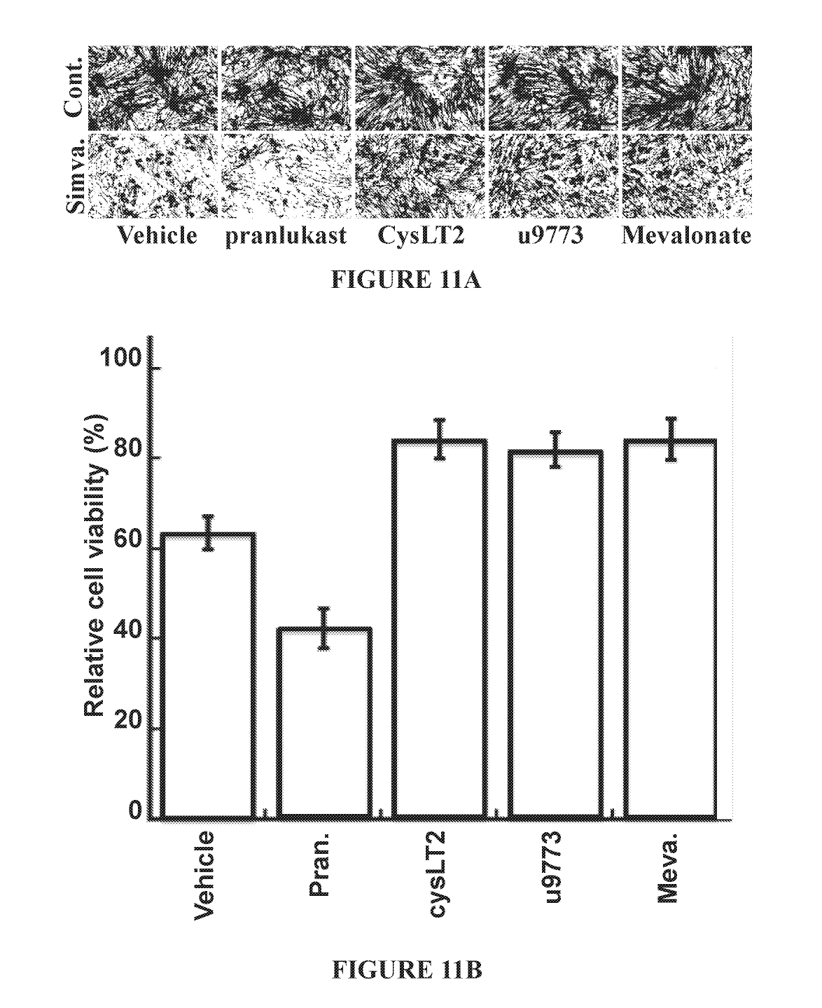

To study the role of the MGST2-LTC4 pathway in statin-triggered cell death, differentiated mouse C2C12 myocytes in 96 well plates were treated with 10 uM simvastatin or vehicle in the presence of either vehicle, BAY-cysLT2 (10 μM) or BAY-u9773 (1 μM) or sodium mevalonate for 5 days. The results show that BAY-cysLT2 as well as BAY-u9773 attenuated cell death triggered by simvastatin. In contrast, the CysLTR1 antagonist pranlukast did not protect these cells from the toxic effects of simvastatin (FIGS. 11A, 11B)

### Example 12—Zileuton Protects Mouse C2C12 Myocyces from Simvastatin-Triggered Cell Death

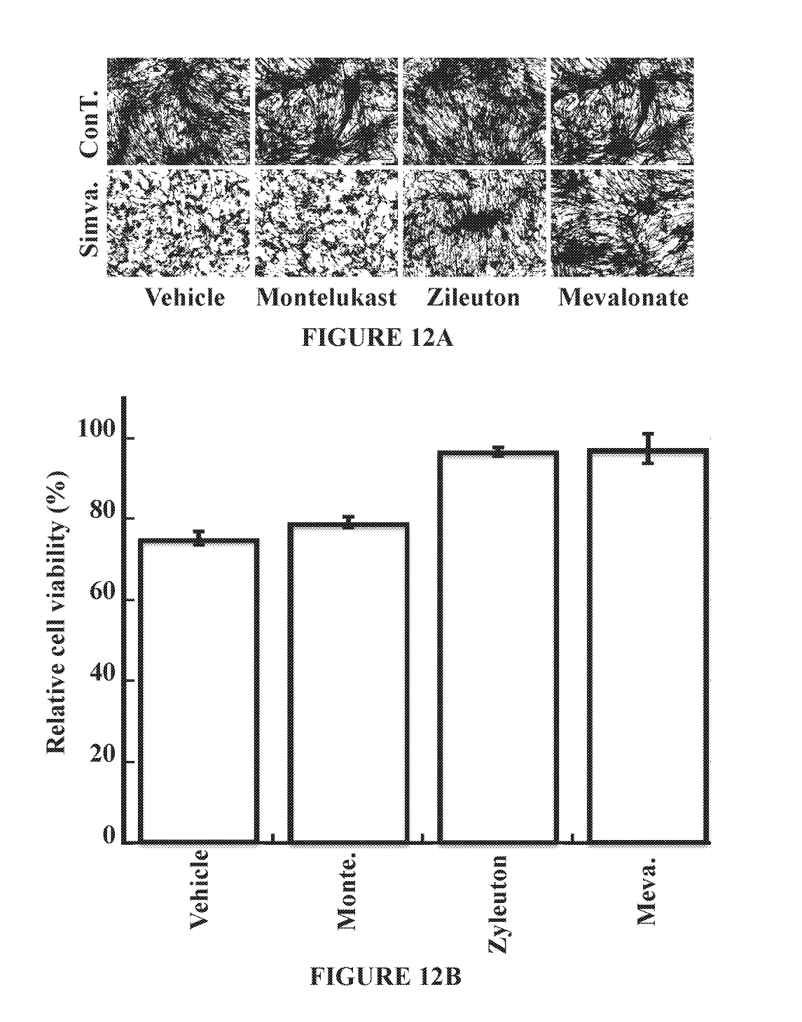

To study the role of the MGST2-LTC4 pathway in statin-triggered cell death, differentiated mouse C2C12 myocytes in 96 well plates were treated with 20 uM simvastatin or vehicle in the presence of either vehicle, zileuton (10 μM) or sodium mevalonate for 4 days. The results show that zileuton attenuated cell death triggered by simvastatin. In contrast, the CysLTR1 antagonist montelukast did not protect these cells from the toxic effects of simvastatin (FIGS. 12A, 12B).

### Example 13—the Effect of Zileuton in Attenuating Statin Therapy Side Effect

The effect of zileuton on patients undergoing statin therapy for treatment of hypercholesterolemia and who had reported suffering from varying levels of muscular pain and fatigue is tested.

Patients are either contacted or attend the clinic at weeks −1, 0, +1, +2 and +4. At the clinic on the first week (WK(−1)) details of the patients' statin medication are recorded and daily doses are noted. Patients are also scored for pain using the McGill Pain Questionnaire (Melzack. R., 1975 “The McGill Pain Questionnaire: Major Properties and Scoring Methods”. Pain 1:277-299, the disclosure of which is included herein in its entirety by way of reference), scales for Pain Rating Index (PRI) and Present Pain Intensity (PPI). In the PRI index, which comprises two scores, higher scores indicate increasing levels of pain. In the PPI index present pain is given a score of 0 to 5, where 0 represents no pain and 5 represents excruciating pain. Patients are also scored at WK(−1) for fatigue using the Fatigue Impact Scale (Fisk, J. D. et al, 1994, “Measuring the functional impact of fatigue: Initial Validation of the Fatigue Impact Scale”. Clinical Infectious Disease, 18 (Suppl 1):S79-83, the disclosure of which is included herein in its entirety by way of reference).

At WK(0) and WK(+4) blood samples are taken from patients to determine individual baseline levels of creatine kinase concentration (CK) (units/L) (as a blood measure of muscle trauma) and HDL-cholesterol or LDL-cholesterol and triglyceride levels. It is to be noted that the normal range for blood creatine kinase concentration is 0-200 units/L.

Commencing at WK(0) and continuing through the study the patients are asked to take a total daily dose of 2400 mg of zileuton, administered orally 4 times a day (600 mg tablets) or twice daily extended release 1200 mg tablets.

To test the effect of zileuton on the patients, questionnaires to determine PRI, PPI and FIS scores are conducted thereafter at weeks +1, +2 and +4. At weeks +1 and +2 the patients were contacted by telephone and asked to mail their self-assessment questionnaires to the clinic.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A pharmaceutical composition comprising:
(i) at least one statin, and
(ii) at least one agent selected from the group consisting of an antagonist of cysteinyl leukotriene receptor 2 (CysLTR2) and an inhibitor of leukotriene C4 (LTC4) biosynthesis.

2. (canceled)

3. The pharmaceutical composition of claim 1, wherein the agent is an antagonist of CysLTR2.

4. The pharmaceutical composition of claim 3, wherein the antagonist of CysLTR2 inhibits the activity of CysLTR2 and the activity of cysteinyl leukotriene receptor 1 (CysLTR1).

5. The pharmaceutical composition of claim 1, wherein the agent is an inhibitor of LTC4 biosynthesis.

6. The pharmaceutical composition of claim 5, wherein the inhibitor of LTC4 biosynthesis inhibits the activity of an enzyme selected from the group consisting of microsomal glutathione S-transferase 2 (MGST2), cytosolic phospholipase A2 (cPLA2), 5-lipoxygenase (5-LO), and 5-lipoxygenase activating protein (FLAP).

7. (canceled)

8. The pharmaceutical composition of claim 1, comprising:
(i) a statin,
(ii) an antagonist of CysLTR2, and
(iii) an inhibitor of LTC4 biosynthesis.

9-11. (canceled)

12. A method for preventing or reducing an adverse side effect induced by a statin in muscle tissue of a subject, comprising the steps of administering to the subject at least one statin and at least one agent selected from the group consisting of an antagonist of CysLTR2 and an inhibitor of LTC4 biosynthesis, wherein the subject is receiving statin therapy and experiencing or at risk of experiencing an adverse side effect induced by the statin.

13-14. (canceled)

15. A kit comprising:
(i) a pharmaceutical composition comprising at least one statin, and
(ii) a pharmaceutical composition comprising at least one agent selected from the group consisting of an antagonist of CysLTR2 and an inhibitor of LTC4 biosynthesis.

16-17. (canceled)

18. The kit of claim 15, wherein the antagonist of CysLTR2 inhibits the activity of CysLTR2 and the activity of CysLTR1.

19. (canceled)

20. The kit of claim 1549, wherein the inhibitor of LTC4 biosynthesis inhibits the activity of an enzyme selected from the group consisting of MGST2, cPLA2, 5-LO, and FLAP.

21. The kit of claim 20, wherein the inhibitor of 5-LO is zileuton or atreleuton.

22. The kit of claim 15, comprising:
(i) a pharmaceutical composition comprising a statin,
(ii) a pharmaceutical composition comprising an antagonist of CysLTR2, and
(iii) a pharmaceutical composition comprising an inhibitor of LTC4 biosynthesis.

23-41. (canceled)

42. A method for preventing or reducing an adverse side effect induced by a statin in muscle tissue of a subject, comprising the step of administering at least one antagonist of CysLTR2 to the subject, wherein the subject is receiving statin therapy and experiencing or at risk of experiencing an adverse side effect induced by the statin.

43. The method of claim 42, wherein the adverse side effect is a statin-induced myopathy.

44. The method of claim 42, wherein the antagonist of CysLTR2 is administered to the subject being treated by the statin prior to the treatment by the statin, at the same time with the treatment by the statin, or together with the treatment by the statin.

45. The method of claim 42, wherein the antagonist of CysLTR2 is administered to the subject being treated by the statin after the treatment by the statin.

46. A method for preventing or reducing an adverse side effect induced by a statin in muscle tissue of a subject, comprising the step of administering to the subject at least one inhibitor of LTC4 biosynthesis, wherein the subject is receiving statin therapy and experiencing or at risk of experiencing an adverse side effect induced by the statin, and
wherein the inhibitor of LTC4 biosynthesis inhibits the activity of an enzyme selected from the group consisting of MGST2, cPLA2, 5-LO, and FLAP.

47. (canceled)

48. The method of claim 46, wherein the inhibitor of LTC4 biosynthesis is administered to the subject being treated by the statin prior to the treatment by the statin, at the same time with the treatment by the statin, or together with the treatment by the statin.

49. The method of claim 46, wherein the inhibitor of LTC4 biosynthesis is administered to the subject being treated by the statin after the treatment by the statin.
